(19) United States
(12) Patent Application Publication
(10) Pub. No.: US 2011/0117069 A1
(43) Pub. Date: May 19, 2011

(54) METHOD FOR ACTIVATING REGULATORY T-CELLS

(75) Inventors: Siegfried Ansorge, Hohenwarthe (DE); Ute Bank, Stassfurt (DE); Uwe Lendeckel, Greifswald (DE); Janine Tadje, Magdeburg (DE); Michael Taeger, Heinrichsberg (DE); Carmen Wolke, Rostock (DE)

(73) Assignee: IMTM GMBH, Magdeburg (DE)

(21) Appl. No.: 12/664,263

(22) PCT Filed: Aug. 21, 2008
(86) PCT No.: PCT/EP2008/006895

§ 371 Date: Dec. 20, 2010

(30) Foreign Application Priority Data
Aug. 21, 2007 (DE) 10 2007 039 429.4

(51) Int. Cl. A61K 35/14; C12N 5/00; A61P 25/00; A61P 17/00; A61P 9/10; A61P 35/00; A61P 35/04; A61P 3/10

(52) U.S. Cl. 424/93.71; 435/375; 435/325

(57) ABSTRACT

The invention relates to a method for activating regulatory t-cells (Treg-cells) of the human or animal body, comprising a step of bringing into contact the regulatory t-cells (Treg-cells) in a suitable liquid medium with one or a plurality of inhibitors of alanyl-amino peptidase (amino peptidase N; APN) and/or with one or a plurality of inhibitors of peptidases with the same substrate specificity to induce a suppressive effect of the regulatory t-cells (Treg-cells).

The present invention relates to a method for activating regulatory T-cells (Treg cells; CD4⁺CD25⁺-cells). In particular, the invention relates to a method for the ex-situ activation of regulatory T-cells using inhibitors of alanyl-aminopeptidase (aminopeptidase N; APN; CD13; EC 3.4.11.2) or using inhibitors of enzymes with analogous enzymatic effect. The invention also relates to the use of inhibitors of alanyl-aminopeptidase and/or of inhibitors of enzymes with analogous enzymatic effect for activating regulatory T-cells.

It is already known that diseases with autoimmune pathogenesis such as type I diabetes mellitus or multiple sclerosis, for example, are based upon an activation and proliferation of autoreactive immune cells (i.e. immune cells directed against the body's own antigens), in particular from autoreactive T-lymphocytes, or the activation and proliferation of such immune cells are indicative of this disease process.

Similar mechanisms are significant in the development of rejection episodes after an organ transplant, except that here it is not primarily “autoantigens” but “foreign antigens” from the donor organ that are responsible for the development of the fatal immune response.

In both cases, i.e. both in autoimmune disorders and in rejection reactions, an undesirable break in “tolerance” of the immune system occurs towards the body's own antigens or those originating from the transplant. The same applies to the excessive immune response in the case of allergies.

Experience in recent years shows that this “tolerance” is actively maintained in the healthy organism by the function and growth of autoreactive T-lymphocytes being actively suppressed. This is achieved by means of a special, suppressive T-cell population, the so-called natural regulatory T-cells (Treg, CD4⁺CD25⁺ cells). Treg cells develop in the thymus [Kawahata K. et al., J. Immunol. 168: 4399-4405, 2002] and make up a proportion of 5 to 10% of the T-cells in peripheral blood. They have an inhibitory effect on CD4⁺ T-cells with the same antigen specificity via direct cell contact. This inhibitory effect is achieved by a strong expression of TGF-β1 in/on the Treg. TGF-β1 is thus presented on the surface of the Treg and binds at the TGF-β1 receptor on autoreactive T-cells, which constitutes a completely new mechanism of action of this strong immunosuppressive cytokine [Nakamura et al., J. Exp. Med. 194: 629-644, 2001].

Treg cells inhibit autoimmunity more efficiently than the immune response to “foreign” antigens [Romagnoli, P. et al., J. Immunol. 168: 1644-1648]. Therefore, restrictions in or losses of function of Treg cells have particular pathogenetic significance in the development of autoimmune disorders. A direct association between the number/function of Treg cells and the manifestation of autoimmune disorders has been identified for type I diabetes [Boudalay, S. et al., Eur. Cytokine Netw. 13: 29-37, 2002; Gregori, S. et al., Diabetes 51: 1367-1374, 2002], for autoimmune encephalomyelitis (animal model for multiple sclerosis) [Furtado, G. C. et al., Immunol. Rev. 182: 122-134, 2001; Muhallab, S. et al., Scand. J. Immunol. 55: 264-273, 2002; Hamilton, N. H. et al., Scand. J. Immunol. 55: 171-177, 2002], for autoimmune ovarian disease (AOD) [Tung, K. S. et al., Immunol. Rev. 182: 135-148, 2001] and also for Crohn's disease [Neurath, M. F. et al., J. Exp. Med. 195: 1129-1143, 2002].

In addition, Treg cells are also responsible for suppressing intestinal or pulmonary inflammation [Singh, B et al., Immunol. Rev. 182: 190-200, 2001; Hori, S. et al., Eur. J. Immunol. 32: 1282-1291, 2002]. The role of Treg cells in suppressing rejection episodes after allogeneic (foreign) organ transplant has also been definitely proved [Kingsley, Cl et al., J. Immunol. 168: 1080-1086, 2002; Taylor, P. A. et al., Blood 99: 3493-3499, 2002; Chiffoleau, E et al., J. Immunol. 169: 5058-5069, 2002]. What all these immunosuppressive functions of Treg cells have in common is that they are distinguished by a high antigen specificity, i.e. each Treg cell clone is directed against a special antigen and inhibits autoreactive T-cells with the same antigen specificity under normal physiological conditions. In the case of autoimmune disorders this function of the Treg cells is lost and autoreactive T-cell clones, as directed against proteins of the pancreatic beta cell in the case of type I diabetes, lead to the occurrence of the autoimmune disorder.

However, this antigen specificity can also be used therapeutically by increasing or recreating the number/function of Treg cells (or dendritic cells activated by these cells) through “antigen-specific” activation of these cells in vivo or ex vivo. The oral application of “antigens” is also suitable for this purpose [Zhang et al., J. Immunol. 167: 4245-4253, 2001]. However, the production of such antigens is technically extremely time-consuming and costly and is restricted to antigen-specific T-cell clones.

The special role of TGF-β1 for the regulation of immunological hyper-reactivity is emphasised by two more recent publications that show that the overproduction of TGF-β1 in CD4⁺ cells caused by genetic manipulation is able to suppress the pathological process. Since in the case of asthma Th2-cells are a decisive factor in the pathogenesis, the function of pathogenic Th2-cell clones can therefore be effectively inhibited by transgenic overproduction of TGF-β1 [Hansen, G. et al., J. Clin. Invest. 105: 61-70, 2000; Thorbecke, G. J. et al., Cytokine Growth Factor Rev. 11: 89-96, 2000]. The disadvantage of these methods for inducing the production of TGF-β1 in CD4⁺ or Treg cells is that they necessitate a genetic manipulation that, on the one hand, is very expensive and, on the other, is unsuitable for a pharmacological application in humans or animals.

The publication DE-A 102 30 381 relates to the use of an inhibitor or a plurality of inhibitors of alanyl-aminopeptidases and/or one or more inhibitors of enzymes with the same substrate specificity for inducing the production of TGF-β1 and the expression of TGF-β1 in and/or on Treg cells and the use for the prevention and/or treatment of autoimmune disorders, allergies, arteriosclerosis and for suppressing transplant rejection.

It has now been surprisingly found that promotion of the suppressive activity of the Treg cells and the expression of TGF-β1 by these Treg cells is attributable to the activating effect of one or more inhibitors of alanyl-aminopeptidases and/or of one or more inhibitors of peptidases with the same substrate specificity on Treg cells. In particular, it has been surprisingly found that it is possible to activate Treg cells outside the human or animal body (ex vivo) by one or more inhibitors of alanyl-aminopeptidases and/or by one or more inhibitors of peptidases with the same substrate specificity and by means of the activated Treg cells, generate a tolerance towards alloantigens and autoantigens in the human or animal body or even overcome an excessive immune response in the body.

Therefore, the invention relates to a method for activating regulatory T-cells (Treg cells) of the human or animal body, comprising a step of bringing the regulatory T-cells (Treg cells) in a suitable liquid medium into contact with one or more inhibitors of alanyl-aminopeptidase (aminopeptidase N; APN) and/or with one or more inhibitors of peptidases with the same substrate specificity by inducing a suppressive effect of the regulatory T-cells (Treg cells).

In particular, the invention relates to a method for the ex-vivo activation of regulatory T-cells (Treg cells) of the human or animal body, comprising the steps:
(a) recovering at least one body fluid comprising Treg cells from at least one human or animal body;
(b) isolating the regulatory T-cells (Treg cells) from the thus obtained human or animal body fluid(s);
(c) bringing the thus isolated and purified regulatory T-cells in a suitable fluid or semi-fluid medium into contact with one or more inhibitors of alanyl-aminopeptidase (aminopeptidase N; APN) and/or with one or more inhibitors of peptidases with the same substrate specificity for an adequate period for activation; and
(d) returning the thus treated regulatory T-cells (Treg cells) in a suitable medium into at least one human or animal body.

Preferred embodiments of this method are claimed in dependent claims 3 to 16.

The invention also relates to activated regulatory T-cells (Treg cells) obtainable using a method that will be described in detail below.

The invention additionally relates to a preparation comprising activated regulatory T-cells (Treg cells), such as produced using the method according to the invention, possibly together with usual supports, auxiliary substances and/or adjuvants.

The invention additionally relates to the use of activated regulatory T-cells (Treg cells) in accordance with the following detailed description and/or the use of preparations comprising such regulatory T-cells (Treg cells) for the prevention, alleviation or therapy of transplant rejection reactions, autoimmune disorders, allergies, bronchial asthma and COPD, diseases of chronic-inflammatory genesis, including arteriosclerosis, neuronal diseases and brain damage, skin diseases, preferably psoriasis, acne or keloids, and other hyperproliferative conditions, fibroses, tumour diseases and sepsis.

Preferred uses are claimed in dependent claims 27 to 38.

The present invention will be explained in further detail below with reference to the figures, wherein:

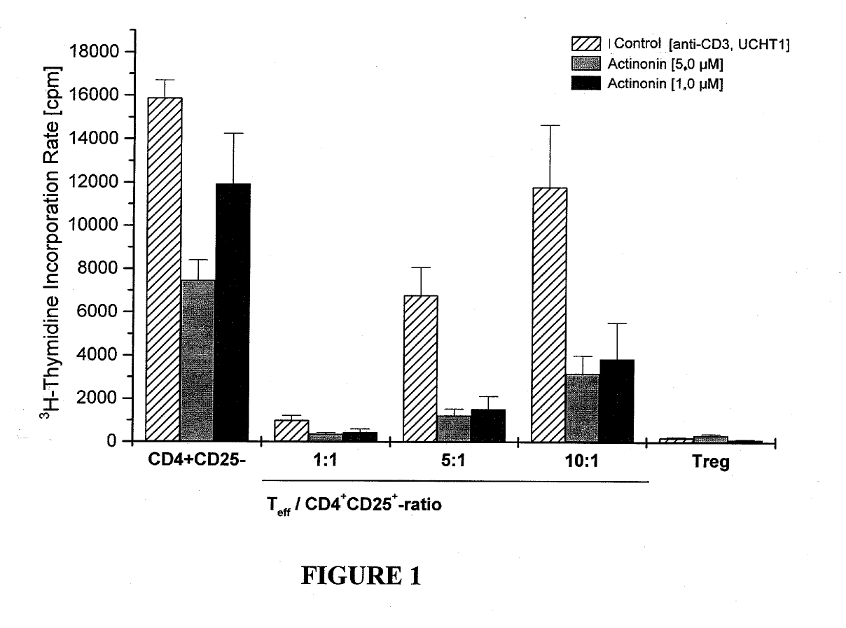

FIG. 1 is a graphic representation that quantitatively demonstrates the activation of human regulatory T-cells in the presence of actinonin as inhibitor of aminopeptidase N;

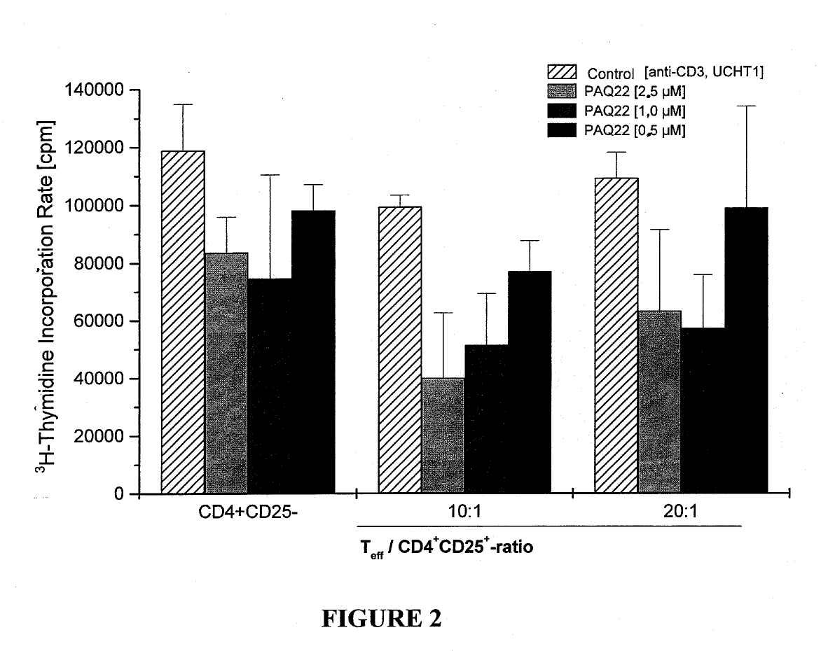

FIG. 2 is a graphic representation that quantitatively demonstrates the activation of human regulatory T-cells in the presence of PAQ22 as inhibitor of cytosolic aminopeptidase (cAAP);

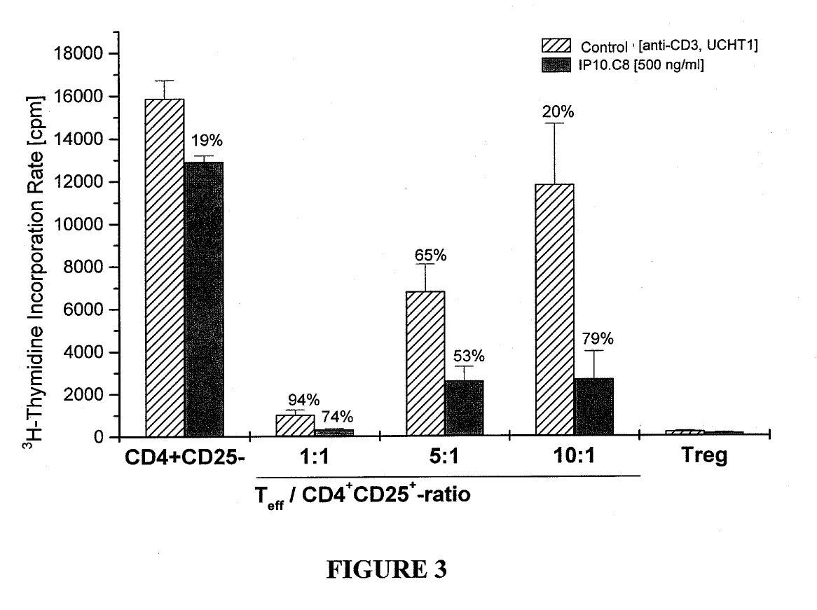

FIG. 3 is a graphic representation that quantitatively demonstrates the activation of human regulatory T-cells in the presence of IP10.C8 as dual inhibitor of alanyl-aminopeptidase (APN) and dipeptidylpeptidase IV (DPIV);

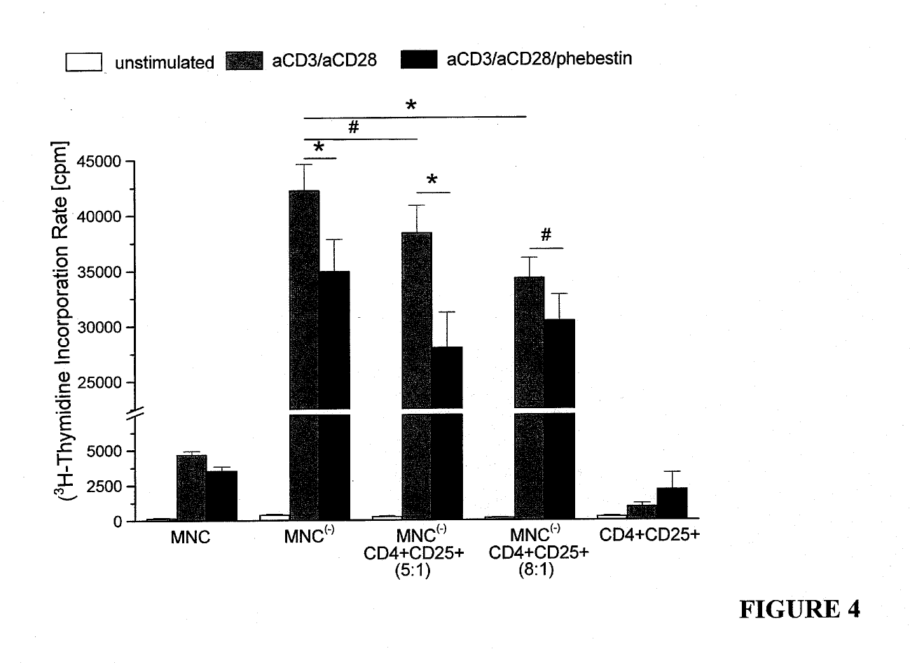

FIG. 4 is a graphic representation that quantitatively demonstrates the activation of murine regulatory T-cells in the presence of phebestin as inhibitor of alanyl-aminopeptidase (APN); and

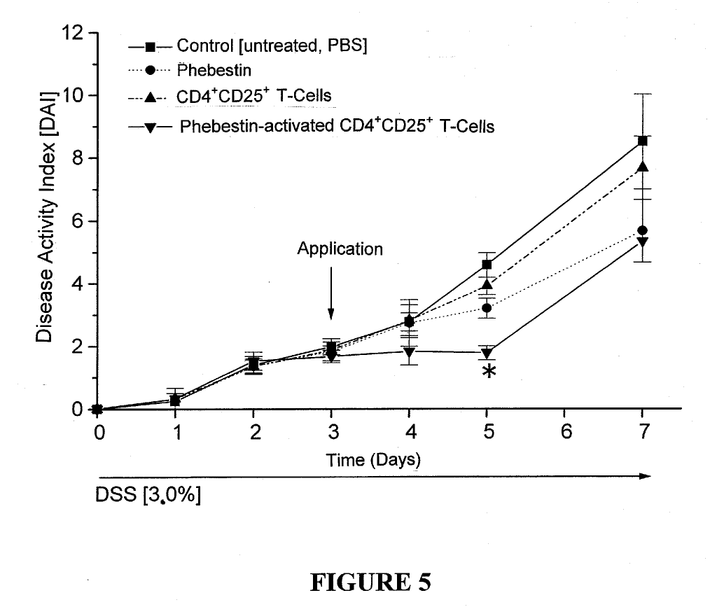

FIG. 5 is a graphic representation that quantitatively demonstrates the effect of regulatory T-cells (Treg cells) activated ex situ with an inhibitor of APN (phebestin) in the colitis model in mice.

The invention will now be explained in further detail with reference to the preferred embodiments and examples, in which the practical application of preferred embodiments is described. However, it should be understood that the invention is not restricted to the preferred embodiments which are merely specified for exemplary explanation.

The invention relates to a method for activating regulatory T-cells (Treg cells, CD4⁺CD25⁺ cells). In the present description and the patent claims, “regulatory T-cells” are understood to be those T-lymphocytes that have the ability to control pathogenic T-cell responses. Treg cells are differentiated in the thymus and are then transported into the periphery of the body. The main task of Treg cells in the human or animal organism is to block the effector function of autoreactive mature T-cells (Sakaguchi, S. et al., J. Immunol. 155: 1151-1164 (1995); Roncarolo, M. G. et al., J. Exp. Med. 193: F5-F9).

The Treg cells are activated in the method according to the present invention. In the present invention and in the patent claims, “activation” is understood to mean that a suppressive effect of the Treg cells is induced, which is expressed in a strong expression of the transforming growth factor β1 (TGF-β1) and the transcription factor FoxP3. According to the invention, the term “activation” also covers a reactivation of Treg cells. This can take place both in vitro and in vivo, for example, after an inactivation of Treg cells under inflammatory conditions, e.g. as a result of long action of inflammatory cytokines.

In the present description and in the patent claims, the term “inhibitor” is understood to mean those compounds of natural origin, synthetic origin or natural origin with synthetic modification that have a regulating effect, in particular a restraining effect on an enzyme or on a group of enzymes. The regulating effect can be based on a wide variety of effects without restrictions having to be made from the aforementioned broad definition of the term “inhibitor”. Preferred inhibitors according to the invention are inhibitors with a restraining effect on enzymes, further preferred on groups of specific enzymes, e.g. inhibitors with a restraining effect on alanyl-aminopeptidase N (APN) and on peptidases with the same substrate specificity as alanyl-aminopeptidase N or inhibitors with a restraining effect on dipeptidylpeptidase IV (DP IV) and on peptidases with the same substrate specificity as dipeptidylpeptidase IV.

A single inhibitor can be used in the step of bringing the regulatory T-cells (Treg cells) in contact with one or more inhibitors of alanyl-aminopeptidase and/or with one or more inhibitors of peptidases with the same substrate specificity. Alternatively, a plurality of inhibitors can be used. The use of one inhibitor is particularly preferred according to the invention. The inhibitor(s) used in the method according to the invention can be one or more inhibitors of alanyl-aminopeptidase. Alternatively, the inhibitor(s) used in the method according to the invention can be one or more inhibitors of peptidases that have the same substrate specificity as alanyl-aminopeptidase. As a further alternative, the inhibitor(s) used can be one or more inhibitors of both alanyl-aminopeptidase and peptidases with the same substrate specificity. In a further alternative embodiment of the method according to the invention, a plurality of inhibitors can be used, of which one or more inhibitors come from the group of inhibitors of alanyl-aminopeptidase and one or more further inhibitors come from the group of inhibitors of peptidases with the same substrate specificity as alanyl-aminopeptidase.

The inhibitor used or—if a plurality of inhibitors are used—the inhibitors used can be an inhibitor (as specified in more detail below in preferred embodiments) of alanyl-aminopeptidase (aminopeptidase N; APN; CD13; EC 3.4.11.2), or the inhibitor can be an inhibitor of a peptidase that has the same substrate specificity as alanyl-aminopeptidase.

The term “inhibitor of alanyl-aminopeptidase (APN)”, as used in the present description and in the patent claims, relates to those substances that can specifically inhibit the enzyme activity of APN and other peptidases with the same substrate specificity. As is known, these inhibitors can belong to different structure types. A joint characteristic of APN inhibitors is their affinity to the active site of APN and peptidases with the same substrate specificity. This is characterised by a Zn²⁺ ion, a zinc-binding motif with the sequence HEXXH-(X18)-E and the exopeptidase motif GXMEN. The essential amino acid residues that are responsible for binding all known APN inhibitors in the active site of APN include E355, H388, E389, H392, E411 and Y477.

These molecular bases of the specific interaction of APN inhibitors with alanyl-aminopeptidase and with peptidases of the same substrate specificity account for the general applicability, irrespective of the special structure of an inhibitor, with respect to the effect and biological role of the inhibitors of APN and the enzymes with the same substrate specificity derived from results of established inhibitors [Xu, W. et al., Curr. Med. Chem.—Anti-Cancer Agents 5: 285-301 (2005); Bouvois, B. et al., Med. Res. Reviews 26:88-130 (2006)].

The term “inhibitors of peptidases with the same substrate specificity (as alanyl-aminopeptidase)” used in the present description and in the patent claims, in the sense of the preceding statements concerning inhibitors, relates to those peptidases whose effect can be defined with inclusion of the highly-preserved zinc binding motif and the exopeptidase motif. Examples of such peptidases are cytosolic aminopeptidase (cAAP; EC 3.4.11.14), aminopeptidase A (APA; EC 3.4.11.7), thyrotropin-releasing hormone-degrading ectoenzyme TRH-DE; EC 3.4.19.6), adipocyte-derived leucine aminopeptidase (A-LAP; EC 3.4.11.x), insulin-regulated aminopeptidase (IRAP; EC 3.4.11.3), aminopeptidase B (APB; EC 3.4.11.6), leukotriene A4 hydrolase (LTA4H; EC 3.3.2.6) and leucocyte-derived arginine aminopeptidase (LRAP; EC 3.4.11.x), without being restricted to the aforementioned examples [Albiston, A. L. et al., Protein and Peptide Letters, vol. XI, No. 5, 491-500 (2004)].

The use of inhibitors of alanyl-aminopeptidase (aminopeptidase N; APN; CD13; EC 3.4.11.2) is particularly preferred according to the invention.

In the method for activating regulatory T-cells (Treg cells) particularly preferred according to the invention on the basis of the unexpected activation results, the step of activation takes place ex-vivo. Within the framework of the present description and in the patent claims this is understood to mean that the method preferred according to the invention is not a method conducted on a (living) human or animal organism. Rather, the activation step is conducted in vitro with substance removed from a living human or animal body and the substance treated according to the invention is then returned to the human or animal body again in a suitable form. As will be seen from the examples based on particularly preferred embodiments illustrated below, this leads to activation results of the Treg cells that are unexpected for the person skilled in the art.

In the first step of the preferred ex-vivo method of the invention for activating regulatory T-cells (Treg cells) of the human or animal body, at least one body fluid that can be used to recover Treg cells, i.e. that comprises Treg cells, is recovered from at least one, preferably from precisely one, human or animal body. A body fluid comprising Treg cells can be recovered from a human or animal body, or a plurality of body fluids comprising Treg cells can be recovered from a human or animal body. This can be performed on or from a living human or animal body in a manner known per se to the skilled person and the method of recovery depends on the body fluid in question. A suitable way of recovering one or more body fluids can be by the secretion of body fluid(s) through the human or animal body (e.g. in the case of exudates) or by the removal of body fluid(s) (e.g. in the case of blood) conducted by a specialist.

In particularly preferred embodiments of the method that do not, however, restrict the invention, one or more body fluids selected from blood, fractions of blood, lymph, exudates or local compartments are recovered from a human or animal body. On a practical basis, a single body fluid selected from the aforementioned body fluids is recovered. If blood is isolated from a human or animal body, then peripheral blood, even further preferred intravenous blood, is preferably selected. Pleura or peritoneum, for example, can preferably be isolated as local compartments. It is particularly preferred if peripheral blood, particularly advantageously intravenous blood, is recovered from a human or animal body. Treg cells are naturally present in intravenous or peripheral blood in concentrations that facilitate the isolation occurring in the following step in a practical manner.

Regulatory T-cells (Treg cells) are isolated in the following method step from the body fluid(s) recovered in the first step of the method according to the invention, i.e. preferably from one of the aforementioned body fluids, in particular from one of the aforementioned body fluids recovered from a human or animal body, further preferred from blood and particularly advantageously from peripheral blood, e.g. from intravenous blood. This can occur using any conceivable procedure for isolating Treg cells known to the skilled person without the invention being subject to any restrictions in this regard. In particular, separation kits for the isolation of

Treg cells are commercially available that reliably enable Treg cells to be isolated from one of the aforementioned body fluids.

Working from the body fluids or from the specific body fluid recovered in the first method step, e.g. from peripheral blood or from intravenous blood, cell fractions that also comprise regulatory T-cells (Treg cells) are separated using suitable separation methods. For example, in a manner known per se mononuclear cells and enriched T-cells from these that comprise Treg cells can be obtained from peripheral donor blood by density-gradient centrifugation using different processes generally known to the person skilled in the art. From the thus obtained cell fraction, regulatory T-cells (Treg cells) can be obtained using separation processes that take into account the properties of the Treg cells, e.g. (without restriction) using cell-specific antibodies linked to magnetic particles. A two-stage magnetic separation has proved advantageous according to the invention. In the first process step thereof, the cell population obtained in the preceding process step can be depleted of CD4⁻ cells with CD4⁺ cells remaining according to the invention. This can be achieved, for example, using commercially available separation kits, e.g. with a CD4⁻ separation kit such as that available from Miltenyi Biotech, Bergisch-Gladbach, Germany. This enables CD4⁺ T-cells with a purity of >95% to be obtained. In the second magnetic column separation step, the remaining cell population is then treated with anti-CD24 MicroBeads (Miltenyi Biotech, Bergisch-Gladbach, Germany) and using CD25 marking CD4⁺CD25⁺ T-cells (regulatory T-cells; Treg cells) are obtained by magnetic column separation. Highly pure regulatory T-cells (Treg cells) can be isolated in this way. In other words: the step of isolating the Treg cells is accompanied by a purification of the Treg cells, i.e. a removal of other cells, cell components or other materials that could obstruct or hinder or even prevent the subsequent activation of the Treg cells. However, the invention is not restricted to this method of isolating and purifying Treg cells that is merely given by way of example.

In the next method step, the thus obtained and purified regulatory T-cells in a suitable fluid medium are brought into contact with one or more inhibitors of alanyl-aminopeptidase (aminopeptidase N; APN) and/or with one or more inhibitors of peptidases with the same substrate specificity for a period that is sufficient for activation. This can occur in any desired manner known and familiar to the skilled person in this field without the invention being subject to any restrictions in this regard.

In a preferred embodiment of the method according to the invention, the bringing into contact of Treg cells and one or more inhibitors is conducted in a suitable fluid medium in accordance with the following specific description relating to the inhibitors.

In the present description and in the patent claims, a “fluid medium” is understood to mean primarily liquid cell culture media such as are commercially available in a variety of forms with and without albumin and serum components. However, this does not constitute a restriction of the invention, but only a concentration on the essential possibilities in practice. Naturally, aqueous media are preferred that have the common feature that they should be physiologically acceptable, i.e. not only for practical reasons of enabling the Treg cells to be subsequently reinfused into a human or animal body, but also with respect to allowing a natural course of the activation process under conditions that come as close as possible to the conditions present in vivo. The media that are particularly preferred for use are therefore selected from physiologically acceptable solutions, cell culture media and nutrient media. It is even further preferred if these media are selected from the group comprising physiologically acceptable aqueous solutions, aqueous cell culture media and aqueous nutrient media. In particularly preferred embodiments of the method according to the invention, serum-free AIMV medium is selected as medium for the activation process. The aforementioned media can be selected individually or in combinations of two or more thereof. The use of media that predominantly contain water or are substantially composed of water is particularly preferred.

In addition, it is preferred according to the present invention to add additives, which are usual in a cell culture and/or cell therapy and are known to a skilled person on the basis of his specialist knowledge, to a fluid medium provided for the activation process. Examples of these are antibiotics, amino acid supplements, vitamin supplements and trace element supplements, either individually or in combination of two or more of the specified substance groups in the medium provided for the activation process or the provided media.

The regulatory T-cells (Treg cells) isolated (and purified) as described above are brought into contact with one or more inhibitors, such as those described in detail above, for a period sufficient for an activation. Where applicable, the addition of interleukin-2, preferably 20 to 100 U/ml, to the culture medium is expedient and/or a stimulation using mitogens such as PHA or PWM and/or using anti-CD3 antibodies is expedient. The incubation period can be easily determined by a skilled person within the scope of defining experiments for a specific system. From experience, this lies in the range of 24 to 48 h without being restricted to this range.

According to the invention, the regulatory T-cells isolated as described above are brought into contact with one or more inhibitors of alanyl-aminopeptidase (aminopeptidase N; APN) and/or with one or more inhibitors of peptidases with the same substrate specificity. One inhibitor can be used in the method according to the invention to activate the Treg cells or a plurality of inhibitors can be used. A single inhibitor can be an inhibitor of alanyl-aminopeptidase or a single inhibitor can be an inhibitor of a peptidase with the same substrate specificity. When using a plurality of inhibitors, two or even more inhibitors can be used in combination. These two or more inhibitors can all be inhibitors of alanyl-aminopeptidase or can all be inhibitors of peptidases with the same substrate specificity, or the two or more inhibitors can be inhibitors partly from the group of inhibitors of alanyl-aminopeptidase and partly from the group of inhibitors of peptidases with the same substrate specificity, or these are inhibitors of alanyl-aminopeptidase and simultaneously also inhibitors of (one or more) peptidases with the same substrate specificity as alanyl-aminopeptidase. It is particularly preferred if an individual inhibitor of one of the two aforementioned groups is used and the use of an inhibitor of alanyl-aminopeptidase is most particularly preferred.

In preferred embodiments of the method according to the invention, one of more known inhibitors from the group actinonin, leuhistin, phebestin, amastatin, bestatin, probestin, arphamenin A, arphamenin B, MR 387A, MR 387B, β-aminothiols, α-aminophosphinic acids and their esters and salts, α-aminophosphonates, α-aminoboric acids, α-aminoaldehydes, hydroxamates of α-amino acids, N-phenylphthalimides, N-phenylhomophthalimides, α-keto amides, thalidomide and derivatives thereof, are used as the at least one inhibitor of alanyl-aminopeptidase and/or as the at least one inhibitor of peptidases with the same substrate specificity. The aforementioned names represent generally usual names familiar to the skilled person of inhibitors or substance groups that can be used as inhibitors in the activation method according to the invention. 100431 The designation “MR 387A” is known to represent the substance

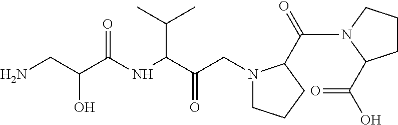

with the systematic name C(2S,3R)-3-amino-2-hydroxy-4-phenylbutano-yl-L-valyl-L-prolyl-L-leucine, and the designation “MR 387B” is known to represent the substance

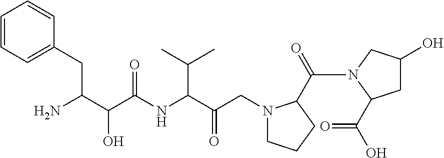

with the systematic name C(2S,3R)-3-amino-2-hydroxy-4-phenylbutano-yl-L-valyl-L-prolyl-(R)-hydroxy-L-proline.

Given purely by way of example and without restricting the present invention hereto, the following compounds, which can be used alone or in combination of a plurality thereof for the activation of Treg cells, can be indicated as suitable inhibitors of alanyl-aminopeptidase:

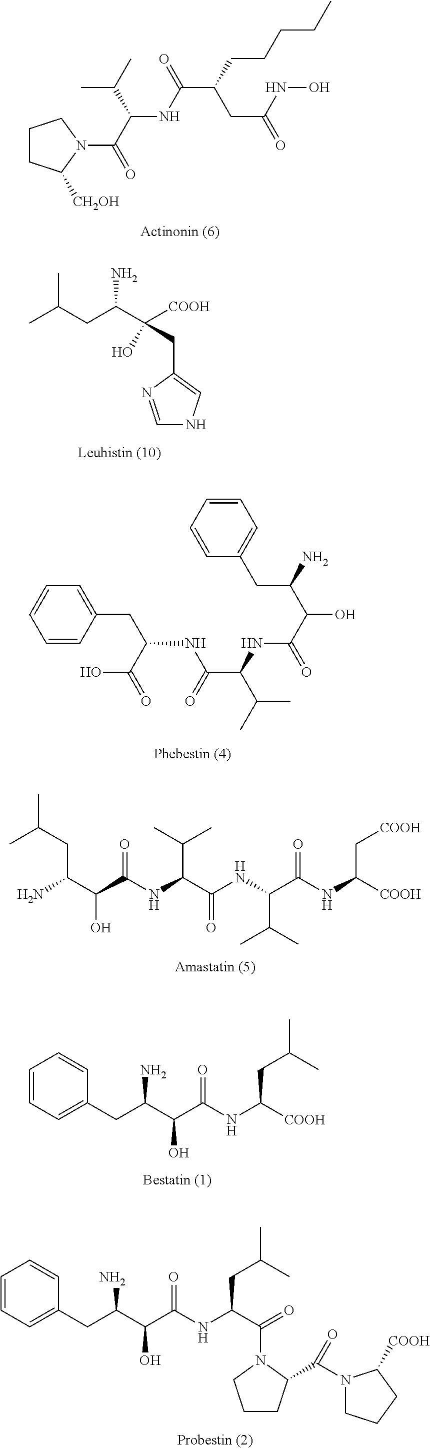

Arphamenin A=5-amino-8-{[amino-(imino-)methyl-]amino-}2-benzyl-4-oxo-octanoic acid

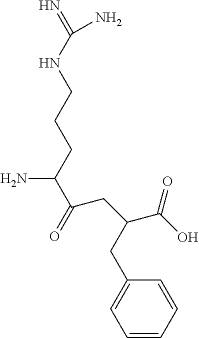

Arphamenin B=5-amino-8-{[amino-(imino-)methyl]amino}-2-(4-hydroxy-benzyl)-4-oxooctanoic acid

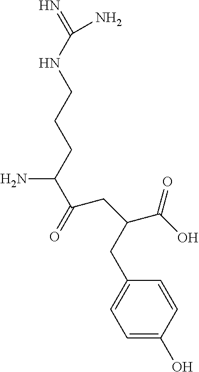

β-Aminothiols:

| | IC₅₀ (nM) |
|---|---|
| Compound | APN |
| 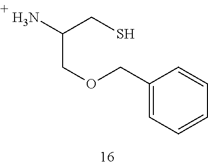 | 56 |
| 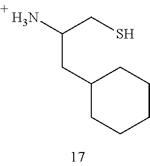 | 45 |
| 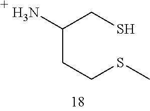 | 11 |
| 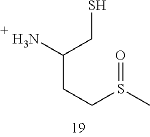 | 20 |
| 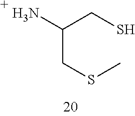 | 20 |
| 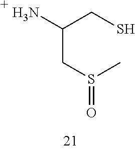 | 21 |
| 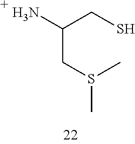 | 22 |
| 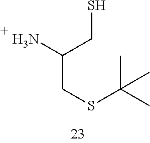 | 40 |
| 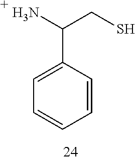 | 25 |
| 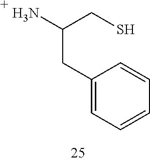 | 30 |
| 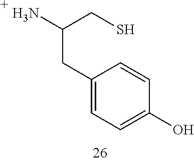 | 45 |
| 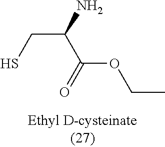 | 350 |
| Compound | Ki (nM) |
| 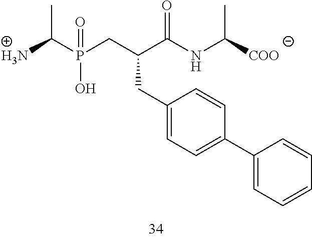 | Ki (APN) = 2.9 Ki (NEP) = 1.2 Ki (ACE) = 120 |
| 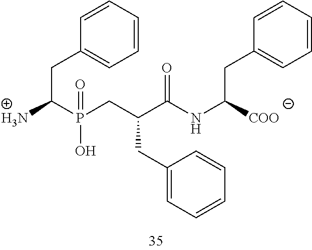 | Ki (APN) = 1.5 Ki (NEP) = 190 |
| 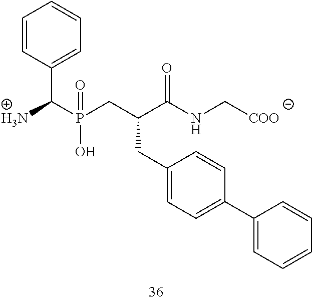 | Ki (APN) = 32.8 Ki (NEP) = 0.94 |
| 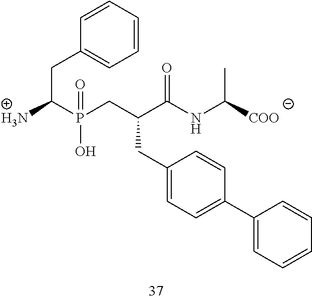 | Ki (APN) = 5.3 Ki (NEP) = 2.2 |
| 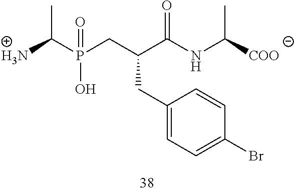 | Ki (APN) = 1.9 Ki (NEP) = 4.9 |
| 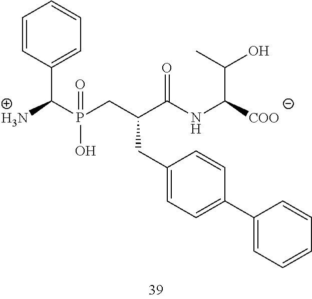 | Ki (APN) = 10.2 Ki (NEP) = 32.5 |
| 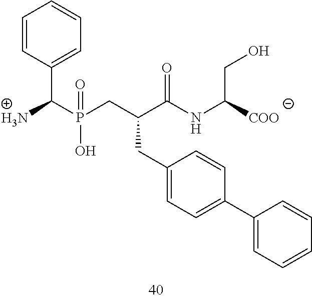 | Ki (APN) = 4.9 Ki (NEP) = 11.8 |
| 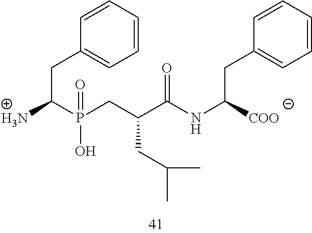 | Ki (APN) = 2.3 Ki (NEP) = 43 |
| 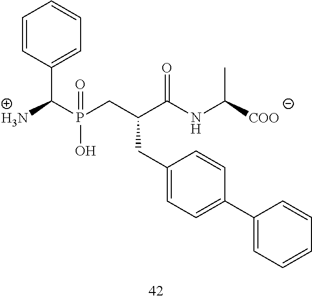 | Ki (APN) = 4.8 Ki (NEP) = 2.0 |
| 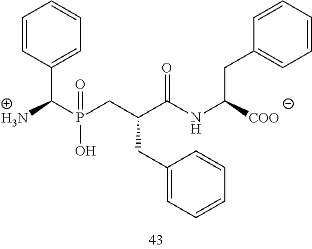 | Ki (APN) = 4.2 Ki (NEP) = 70 |

α-Aminoboric acids:

| | IC₅₀, μM | IC₅₀, μM |
|---|---|---|
| Compound | (LAP) | (APN) |
| 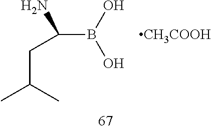 | 0.25 | nd^{a} |
| 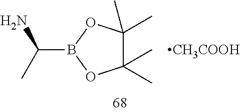 | 0.35 | 0.07 |
| 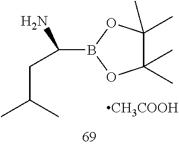 | 0.25 | 0.074 |
| 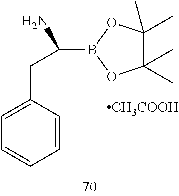 | 0.2 | 0.05 |
| 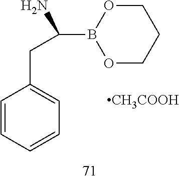 | 0.2 | nd |

α-Aminoaldehydes:

| Compound | Ki, (μM) |
|---|---|
| 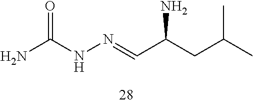 | 230 |
| 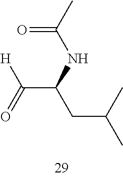 | 430 |
| 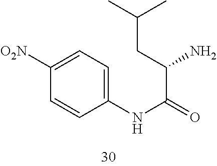 | 520 |
| 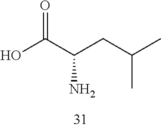 | 2950 |
| 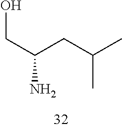 | 4400 |
| 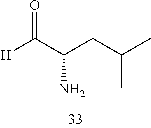 | 0.76 |

N-Phenylphthalimides and -homophthalimides:

| Compound | IC₅₀^{a}, (μM) |
|---|---|
| 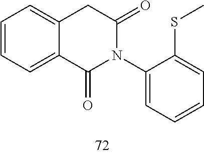 | 0.90 |
| 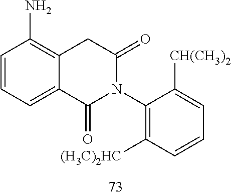 | 5.4 |
| 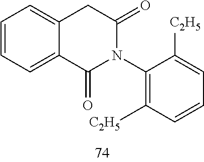 | 0.12 |
| 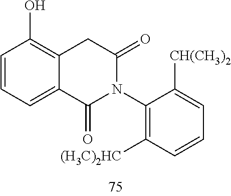 | 4.3 |

α-Keto amides:

| | Ki, (μM) | | | |
|---|---|---|---|---|
| | Cytosolic | | Argininyl | |
| Compound | AP | APN | AP | Ref |
| 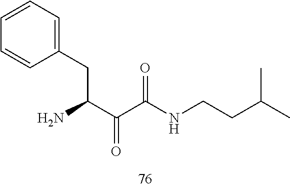 | 1.0 | 2.5 | 1.5 | [87] |
| 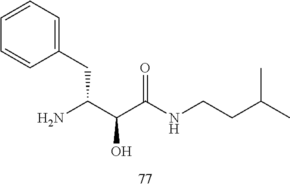 | 0.51 | 20 | 39 | [88] |
| 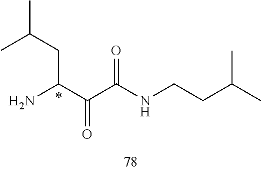 | >15 (R) 1.9 (S) | 18.6 (R) 10.5 (S) | 6.5 (R) 3.2 (S) | [87] |
| 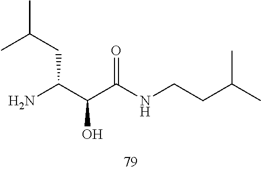 | 5.4 | 24 | >300 | [88] |

The compounds are given in detail in the publication “Xu, W. et al.; Curr. Med. Chem. Anti-Cancer Agents 5: 281 to 301 (2005)” and are described with respect to their inhibitory effect on alanyl-aminopeptidase. The content of this publication is herewith incorporated into the disclosure of the present application by reference.

It is more preferred to use one of more known inhibitors from the group α-keto amides, α-aminophosphinic acids, N-phenylhomophthalimides and α-aminophosphonates as the at least one inhibitor of alanyl-aminopeptidase and/or as the at least one inhibitor of peptidases with the same substrate specificity in the method according to the invention for activating regulatory T-cells (Treg cells). If α-keto amides are used, a compound from the group of 3-amino-2-oxo-4-phenylbutyric acid amides can preferably be used. If α-aminophosphinic acids are used, the use of D-Phe-y[PO(OH)—CH₂]-Phe-Phe is particularly preferred. If N-phenylhomophthalimides are used, the use of PAQ-22 is particularly preferred. If α-aminophosphonates are used, the use of RB3014 and/or phebestin is particularly preferred. Of the specified preferred compounds, the use of PAQ-22, RB3014 and/or phebestin is most particularly preferred as the at least one inhibitor of alanyl-aminopeptidase and/or as the at least one inhibitor of peptidases with the same substrate specificity. PAQ-22 or a plurality of known inhibitors comprising PAQ-22 (i.e. one of which being PAQ-22) can preferably be used as the at least one inhibitor of alanyl-aminopeptidase and/or as the at least one inhibitor of peptidases with the same substrate specificity with particular advantage while retaining extraordinarily good activation results for the Treg cells to be activated. In this case, the abbreviated name RB3014 represents the substance

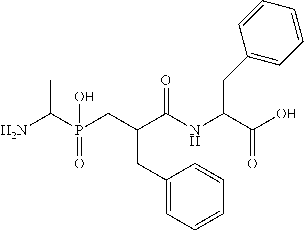

with the systematic name 2-{3 [(1-aminoethyl-)hydroxyphosphinoyl]-2-benzyl-propionylamino-}3-phenylpropionic acid. PAQ-22 stands for the substance

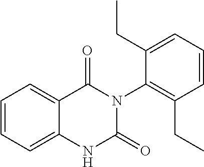

with the systematic name 3-(2,6-diethylphenyl-)quinazoline-2,4(1H3H)-dione.

In a further embodiment likewise preferred according to the invention, one or more known inhibitors from the group of dual inhibitors of alanyl-aminopeptidase or of peptidases with the same substrate specificity and of dipeptidylpeptidases (IV) or of peptidases with the same substrate specificity from the group of compounds of the general formulae (1) and (2) are used as the at least one inhibitor of alanyl-aminopeptidase and/or as the at least one inhibitor of peptidases with the same substrate specificity

A-B-D-B′-A′ (1) and

A-B-D-E (2),

wherein
A and A′ can be the same or different and stand for the radical

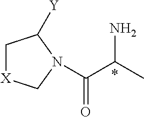

wherein X stands for S, O, CH₂, CH₂CH₂, CH₂O or CH₂NH and Y stands for H or CN and * represents a chiral carbon atom preferably in the S- or L-configuration;
B and B′ can be the same or different and stand for an unsubstituted or substituted, unbranched or branched alkylene radical, cycloalkylene radical, aralkylene radical, heterocycloalkylene radical, heteroarylalkylene radical, aryl-amidoalkylene radical, heteroarylamidoalkylene radical, containing or not containing O, N or S, unsubstituted or mono- or polysubstituted arylene radical or heteroarylene radical with one or more five-, six- or seven-membered ring(s);
D stands for —S—S— or —Se—Se—; and
E stands for the group —CH₂—CH (NH₂)—R⁹ or —CH₂—*CH (NH₂)—R⁹, wherein R⁹ stands for an unsubstituted or substituted, unbranched or branched alkyl radical, cycloalkyl radical, aralkyl radical, heterocycloalkyl radical, heteroarylalkyl radical, arylamidoalkyl radical, heteroarylamidoalkyl radical, containing or not containing O, N or S, unsubstituted or mono- or polysubstituted aryl radical or heteroaryl radical with one or more five-, six- or seven-membered ring(s), and * represents a chiral carbon atom preferably in the S- or L-configuration;
or acid addition salts thereof with organic and/or inorganic acids.

In the present description and in the patent claims, the term “dual inhibitors” is understood to mean inhibitors that are inhibitors of alanyl-aminopeptidase and/or inhibitors of peptidases with the same substrate specificity (as defined above) as well as inhibitors of dipeptidylpeptidases IV (DP IV; CD26; EC 3.4.14.5) and/or inhibitors of peptidases with the same substrate specificity.

In the present description and in the patent claims, the term “inhibitors of dipeptidylpeptidases IV (DP IV)” is understood to mean those substances that are able to specifically inhibit the enzyme activity of DP IV and other peptidases with the same substrate specificity. These DP IV inhibitors can belong to different structure types in this case. A joint characteristic of these inhibitors is their affinity to the active site of DP IV and peptidases with the same substrate specificity. This molecular region of DP IV and other peptidases with the same substrate specificity is characterised by amino acid residues S630, D708, H740 (“catalytic triads”), E205, E206 and Y547. Moreover, residues Y666, F357 and R358 belong to the inhibitor-binding amino acid residues.

These molecular bases of the specific interaction of DP IV inhibitors and the inhibitors of peptidases of the same substrate specificity account for the general applicability, irrespective of the special structure of the inhibitors, with respect to the effect and biological role of these inhibitors derived from results of binding established inhibitors [cf. Sedo, A. et al., Biochimica et Biophysica Acta 1550: 107-116 (2001)].

The above publication also demonstrates numerous examples of peptidases that have the same substrate specificity as dipeptidylpeptidase IV (in a similar sense to the peptidases with the same substrate specificity as APN already defined above). These include, for example, (without restriction) fibroblast-activating protein a, dipeptidylpeptidase IV β, dipeptidyl-aminopeptidase-like protein (DPPX), NAALADase (N-acetylated α-linked acidic dipeptidase), QPP (quiescent cell proline dipeptidase), dipeptidylpeptidase II (DP II), attractin (mahogany protein), dipeptidylpeptidase 8 (DP 8), dipeptidylpeptidase 9 (DP 9).

In the compounds of the above general formulae (1) and (2) that are dual inhibitors in the sense of the above definition, A and A′, which can be the same or different, stand for a radical

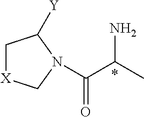

wherein X stands for S, O, CH₂, CH₂CH₂, CH₂O or CH₂NH and Y stands for H or CN and * represents a chiral carbon atom. Compounds of the general formula (1), in which A and A′ are the same, as well as compounds of the general formulae (1) and (2), in which in the above radical represented by A X stands for S, CH₂ or CH₂CH₂ and/or Y stands for H or CN, are particularly preferred according to the invention.

In further preferred embodiments of the invention, such compounds of the general formulae (1) and (2) represent prodrugs to inhibitors particularly active in the activation of Treg cells, in which the chiral carbon atom referred to by * has an S- or L-configuration.

In the description and in the patent claims, the term “prodrug” is understood to relate to naturally occurring or synthetic or naturally occurring but synthetically modified compounds, from which other compounds can be chemically derived or derivatised under certain conditions, preferably under physiological or pathological conditions or under conditions of a desired chemical reaction (such as e.g. the activation of Treg cells), wherein these other compounds develop a chemical or pharmacological efficacy that differs qualitatively and/or quantitatively from that of the starting substance (the “prodrug”). Thus, inhibitor prodrugs are understood to be compounds of natural or synthetic origin, or natural but synthetically modified compounds that, preferably under physiological or pathological conditions or conditions of a desired chemical reaction, can purposefully react to form new substances with inhibitory efficacy. This does not exclude an ability of these prodrugs as such to develop pharmacological efficacy (for example, to inhibit one of the two aforementioned enzymes) already before conversion into drugs with specific pharmacological (e.g. inhibitory) efficacy. Conditions for the conversion of prodrugs into drugs for mammals or specifically humans can be such as those that regularly occur in the physiological environment of a mammal, e.g. a human, or in the body of a mammal, e.g. a human. Alternatively, such physiological conditions can only be present under specific conditions, e.g. a specific physiological state such as conditions determining a clinical picture, for example, in a mammal such as a human, for example, or they can be induced or adapted by external action, e.g. (without restriction) by drug action, on the organism of a mammal such as e.g. the organism of a human, or by creating specific chemical reaction conditions.

In the compounds of the above general formulae (1) and (2), B and B′ can be the same or different and stand for an unsubstituted or substituted, unbranched or branched alkylene radical, cycloalkylene radical, aralkylene radical, heterocycloalkylene radical, heteroarylalkylene radical, arylamidoalkylene radical, heteroarylamidoalkylene radical, containing or not containing 0, N or S, unsubstituted or mono- or polysubstituted arylene radical or heteroarylene radical with one or more five-, six- or seven-membered ring(s).

In the present description and in the patent claims, the term “alkyl radical” is understood to relate to a monovalent straight-chain (unbranched) or branched radical comprising carbon atoms bound to one another via single bonds with hydrogen atoms bound to the carbon atoms. Therefore, in the sense of the present invention, alkyl radicals are saturated monovalent hydrocarbon residues. The alkyl radicals in the compounds of the general formulae (1) and (2) preferably comprise 1 to 18 carbon atoms and are thus selected from the radicals methyl, ethyl, n-propyl, i-propyl and the numerous different straight-chain and branched isomers of the radicals butyl, pentyl, hexyl, heptyl, octyl, nonyl, decyl, undecyl, dodecyl, tridecyl, tetradecyl, pentadecyl, hexadecyl, heptadecyl and octadecyl. Straight-chain and branched alkyl radicals with 1 to 12 carbon atoms are particularly preferred and straight-chain and branched alkyl radicals with 1 to 6 carbon atoms are still further preferred. The radicals methyl, ethyl, n-propyl, i-propyl, n-butyl, i-butyl, sec-butyl and tert-butyl are most preferred.

Accordingly, in the present description and in the patent claims, the terms “alkenyl radical” and “alkinyl radical” are understood to relate to monovalent straight-chain (unbranched) or branched radicals comprising carbon atoms bound to one another via single bonds and at least one double bond or triple bond to any desired, but defined location in the molecule with hydrogen atoms bound to the remaining bonds of carbon atoms that have at least 2 carbon atoms and up to 18 carbon atoms. Vinyl radicals or allyl radicals are preferred examples of such radicals. However, radicals having multiple carbon-carbon bonds are not restricted to the two aforementioned radicals.

In the present description and in the patent claims, the term “alkylene radical” is understood to relate to divalent straight-chain (unbranched) or branched radicals comprising carbon atoms bound to one another via single bonds with hydrogen atoms bound to the carbon atoms. Therefore, in the sense of the present invention, alkylene radicals are saturated divalent hydrocarbon residues. The alkylene radicals in the compounds of the general formulae (1) and (2) preferably comprise 1 to 18 carbon atoms and are thus selected from the radicals methylene, ethylene, n-propylene, 2,2-propylene, 1,2-propylene and the numerous different straight-chain and branched isomers of the radicals butylene, pentylene, hexylene, heptylene, octylene, nonylene, decylene, undecylene, dodecylene, tridecylene, tetradecylene, pentadecylene, hexadecylene, heptadecylene and octadecylene. Straight-chain and branched alkylene radicals with 1 to 12 carbon atoms are particularly preferred and straight-chain and branched alkylene radicals with 1 to 6 carbon atoms are still further preferred. The radicals methylene, ethylene, n-propylene, 2,2-propylene, 1,2-propylene and the numerous different butylene positional isomers are most preferred.

In the alkyl radicals and/or the alkylene radicals, which can be part of the compounds of the general formulae (1) and (2) according to the invention, the chains comprising carbon atoms can be interrupted by —O— atoms, —N— atoms or —S— atoms. Therefore, instead of one or more —CH₂— groups, one or more groups from the group —O—, —NH— and —S— can be located in the course of the chain, wherein two of the groups —O—, —NH— and/or —S— do not usually follow one another in the chain. The one of more groups —O—, —NH— or —S— can be inserted at any desired locations in the molecule in this case. Such a group is preferably contained in the molecule when such a hetero group is present.

According to the invention, in a further embodiment, both straight-chain and branched alkyl or alkylene radicals in the compounds of the general formulae (1) and (2) can be substituted with one or more substituents, preferably with one substituent. The substituent(s) can stand at any desired positions of the skeleton formed from carbon atoms, and (without restricting the invention thereto) can preferably be selected from the group comprising halogen atoms such as fluorine, chlorine, bromine and iodine, particularly preferred chlorine and bromine, alkyl groups with 1 to 6 C atoms, particularly preferred alkyl groups with 1 to 4 C atoms, alkoxy groups with 1 to 5 C atoms in the alkyl radical, preferably 1 to 3 C atoms in the alkyl radical, amino groups, carbonyl groups and carboxyl groups that are unsubstituted or substituted with one or two alkyl radicals respectively independently of one another with 1 to 6 C atoms, preferably 1 to 3 C atoms. The latter can also be present in the form of salts or esters with alcohols with 1 to 6 carbon atoms in the alkyl radical. The term “carboxyl groups” therefore includes groups with the basic structure —COO⁻ M⁺ (with M=monovalent metal atom such as e.g. alkali metal atom or corresponding equivalent of a multivalent metal atom such as e.g. half equivalent of a divalent metal atom such as e.g. an alkaline earth metal atom), or the basic structure —COORₓ (with Rₓ=alkyl group with 1 to 6 carbon atoms). The substituted alkyl groups are selected from the alkyl groups defined in detail above, and it is most particularly preferred if they are methyl groups, ethyl groups, n-propyl groups, i-propyl groups, n-butyl groups, i-butyl groups, sec-butyl groups or tert-butyl groups. Alkoxy groups are alkyl groups in the above-defined sense that are bound via a bridge —O— atom to the skeleton formed from carbon atoms. They are preferably selected from the group comprising the radicals methoxy, ethoxy, n-propoxy, i-propoxy, n-butoxy, i-butoxy, sec-butoxy and tert-butoxy. Amino groups are groups with the basic structure —NRₓR_{y}, wherein the radicals Rₓ and R_{y}, independently of one another, can stand for hydrogen or alkyl groups (in accordance with the above definition) with 1 to 6 carbon atoms, particularly preferred with 1 to 3 C atoms, wherein the radicals Rₓ and R_{y} can be the same or differ from one another. Particularly preferred amino groups as substituents are the groups —NH₂, —NH(CH₃), —N(CH₃)₂, —NH(C₂H₅) and —N(C₂H₅)₂. The term “amino groups” also covers groups of the above-defined structure, which are present as quaternised ammonium ions, either as a result of salt formation with organic acids or inorganic acids (i.e. radicals of the structure RₓR_{y}R_{z}N⁺Q⁻, wherein Rₓ, R_{y} and R_{z} can be the same or different, are preferably the same, and can have the meanings defined above for Rₓ and R_{y}, and at least one of the radicals is hydrogen from the quaternisation with organic or inorganic acid and Q stands for an acid residue of the organic or inorganic acid) or as a result of salt formation with suitable quaternising reagents known to the person skilled in this field such as with alkyl halides, for example (no restriction hereto).

In the present description and in the patent claims, the term “cycloalkyl” stands for unsubstituted or substituted monovalent radicals comprising —CH₂— groups connected to form closed rings. According to the invention, these groups can preferably contain three to eight atoms in the ring and can either be composed exclusively of carbon atoms or contain one or more heteroatoms, which is/are selected from —O—, —S— and —NRₓ—, wherein Rₓ stands for hydrogen or an alkyl radical (as defined above) with 1 to 6 carbon atoms. In the cases where heteroatoms are bound into the rings, these—where a plurality of heteroatoms are present—can be the same or different. In the case where heteroatoms are present, one heteroatom is preferably bound into the ring. The radicals particularly preferred among the purely carbocyclic rings are cyclopentyl, cyclopentenyl, cyclopentadienyl, cyclohexyl, cyclohexenyl, cyclohexadienyl, cycloheptyl, cycloheptenyl, cycloheptadienyl and cycloheptatrienyl. In further embodiments of the invention, examples of cycloalkyl radicals containing heteroatoms, also referred to as heterocycloalkyl radicals, are the radicals tetrahydrofuranyl, pyrrolidinyl, pyrazolidinyl, imidazolidinyl, piperidinyl, piperazinyl and morpholinyl.

Possible substituents on these carbocyclic or heterocyclic cycloalkyl radicals can preferably be selected from the above group of substituents for linear alkyl groups (without restricting the invention thereto). Particularly preferred substituents for cycloalkyl groups are the substituents —Cl, —Br, methyl, ethyl, n-propyl, i-propyl, n-butyl, i-butyl, sec-butyl or tert-butyl, methoxy, ethoxy, n-propoxy, i-propoxy; n-butoxy, i-butoxy, sec-butoxy and tert-butoxy, —NH₂, —NH(CH₃), —N(CH₃)₂, —NH(C₂H₅) and —N(C₂H₅)₂, carbonyl and carboxyl

In the present description and in the patent claims, the term “cycloalkylene” stands for unsubstituted or substituted divalent radicals comprising —CH₂— groups connected to form closed rings. According to the invention, these groups can preferably contain three to eight atoms in the ring and can either be composed exclusively of carbon atoms or contain one or more heteroatoms, which is/are selected from —O—, —S— and —NRₓ—, wherein Rₓ stands for hydrogen or an alkyl radical (as defined above) with 1 to 6 carbon atoms. The radicals particularly preferred among the purely carbocyclic rings are cyclopentylene, cyclopentenylene, cyclopentadienylene, cyclohexylene, cyclohexenylene, cyclohexadienylene, cycloheptylene, cycloheptenylene, cycloheptadienylene and cycloheptatrienylene. The heterocyclic groups defined above in the case of the cycloalkyl radicals can also occur as divalent radicals in the compounds of the general formulae (1) and (2) as “B” groups, and particularly preferred are those cyclic divalent radicals, in which an —O— or —NRₓ— group is bound into the ring. In these cases, both valences are localised at any desired C atoms in the ring. It is preferred if one heteroatom or two heteroatoms are bound into the ring, and in particularly preferred embodiments those groups are the divalent radicals derived from tetrahydrofuran, pyrrolidine, pyrazolidine, imidazolidine, piperidine, piperazine and morpholine.

Possible substituents on these carbocyclic or heterocyclic cycloalkylene radicals can preferably be selected from the above group of substituents for linear alkyl groups (without restricting the invention thereto). Particularly preferred substituents for cycloalkylene groups are the substituents —Cl, —Br, methyl, ethyl, n-propyl, i-propyl, n-butyl, i-butyl, sec-butyl or tert-butyl, methoxy, ethoxy, n-propoxy, i-propoxy; n-butoxy, i-butoxy, sec-butoxy and tert-butoxy, —NH₂, —NH(CH₃), —N(CH₃)₂, —NH(C₂H₅) and —N(C₂H₅)₂, carbonyl and carboxyl.

Within the framework of the present description and in the patent claims, “aryl radical” is understood to mean a monovalent hydrocarbon residue, which can be unsubstituted or substituted, derived from a cyclic molecule with an aromatic character (4n+2 π-electrons delocalised in ring orbitals). The ring structure of such an aryl radical can be a five-, six- or seven-membered ring structure with a ring or a structure formed from two or more rings bonded to one another (annellated), wherein the annellated rings can have the same or a different number of ring members, in particular of C atoms. In the case of systems composed of a plurality of rings annellated to one another, benzo-condensed rings are preferred, i.e. ring systems in which at least one of the rings is an aromatic six-membered ring (phenyl ring) composed only of C atoms. Typical, but not restrictive examples of aryl radicals are cyclopentadienyl radicals (C₅H₅⁻) (as five-membered ring), phenyl radicals (as six-membered rings), cycloheptatrienyl radicals (C₇H₇⁺) (as seven-membered ring), naphthyl radicals (as ring system comprising two annellated six-membered rings) and also monovalent radicals derived from anthracene and phenanthrene (three annellated six-membered rings). The aryl radicals most preferred according to the invention are phenyl and naphthyl radicals. Possible substitutes on these carbocyclic aryl radicals can preferably be selected from the above group of substituents for linear alkyl groups without restricting the invention to these substituents. Particularly preferred substituents for aryl groups are the substituents —Cl, —Br, methyl, ethyl, n-propyl, i-propyl, n-butyl, i-butyl, sec-butyl or tert-butyl, methoxy, ethoxy, n-propoxy, i-propoxy; n-butoxy, i-butoxy, sec-butoxy, tert-butoxy, —NH₂, —NH(CH₃), —N(CH₃)₂, —NH(C₂H₅) and —N(C₂H₅)₂, carbonyl and carboxyl. One or more such substituents, which can be the same or different, can be bonded to an aryl radical according to the present invention. The substituted position(s) on the aryl ring (system) can be selected as desired.

A comparable definition to that in the case of aryl radicals applies to the term “arylene radical” within the framework of the present description and the patent claims. This is understood to relate to a divalent radical, the fundamental structure and selection and substituent(s) of which are comparable to the above details for the definition of the “aryl radicals”, except that this is a divalent radical that can be bonded to any two carbon atoms of the ring.

Within the framework of the present description and the patent claims, “heteroaryl radical” is understood to relate to an aryl radical (in the sense of the above definition), in the ring structure of which a heteroatom or a plurality of heteroatoms, preferably from the group O, N or S, is/are contained without the aromatic character of the molecule being lost thereby. Heteroaryl radicals according to the invention can be unsubstituted or substituted. The ring structure of such a heteroaryl radical can be a five-, six- or seven-membered ring structure with a ring or a structure formed from two or more rings bonded to one another (annellated), wherein the annellated rings can have the same or a different number of ring members. The heteroatom(s) can be present alone in one or also in several of the rings of the ring system. The heteroaryl radicals preferably comprise one or two rings. In the case of systems composed of a plurality of rings annellated to one another, benzo-condensed rings are particularly preferred, i.e. ring systems in which at least one of the rings is an aromatic carbocyclic (i.e. composed only of C atoms) six-membered ring. Particularly preferred heteroaryl radicals are selected from furanyl, thiophenyl, pyridyl, indolyl, cumaronyl, thionaphthenyl, quinolinyl (benzopyridyl), quinazolinyl (benzopyrimidinyl) and quinoxylinyl (benzopyrazinyl).

Possible substitutes on these heteroaryl radicals can preferably be selected from the above group of substituents for linear alkyl groups without restricting the invention to these substituents. Particularly preferred substituents for heteroaryl groups are the substituents —Cl, —Br, methyl, ethyl, n-propyl, i-propyl, n-butyl, i-butyl, sec-butyl or tert-butyl, methoxy, ethoxy, n-propoxy, i-propoxy; n-butoxy, i-butoxy, sec-butoxy, tert-butoxy, —NH₂, —NH(CH₃), —N(CH₃)₂, —NH(C₂H₅) and —N(C₂H₅)₂, carbonyl and carboxyl. One or more such substituents, which can be the same or different, can be bonded to a heteroaryl radical according to the present invention. The substituted position(s) on the heteroaryl ring (system) can be selected as desired.

A comparable definition to that in the case of heteroaryl radicals applies to the term “heteroarylene radical” within the framework of the present description and the patent claims. This is understood to relate to a divalent radical, the fundamental structure and selection and substituent(s) of which are comparable to the above details for the definition of the “heteroaryl radicals”, except that this is a divalent radical that can be bonded to any two carbon atoms of the ring or ring system or can also be bonded to a nitrogen atom.

Within the framework of the present description and the patent claims, the following terms used: “aralkyl radical”, “heteroarylalkyl radical”, “heterocycloalkyl radical”, “arylamidoalkyl radical” and “heteroarylamidoalkyl radical” mean alkyl radicals (or—more precisely—alkylene radicals) in the sense of the above general and specific definition, which are substituted at one of their bonds with an aryl radical (in accordance with the above general and specific definition), heteroaryl radical (in accordance with the above general and specific definition), heterocyclyl radical (in accordance with the above general and specific definition of the cycloalkyl radicals substituted with heteroatoms), arylamido radical (in accordance with the above general and specific definition) or heteroarylamido radical (in accordance with the above general and specific definition). These radicals can be unsubstituted or substituted.

In preferred embodiments of the invention, aralkyl radicals are radicals, in which the aryl radical is a phenyl radical, substituted phenyl radical, naphthyl radical or substituted naphthyl radical, and the alkyl(ene) group is straight-chain or branched and has 1 to 6 carbon atoms. The radicals benzyl, phenethyl, naphthylmethyl and naphthylethyl can be used particularly advantageously as aralkyl radical, and of these benzyl radicals are most particularly preferred.

Possible substituents on the aryl groups of the aralkyl radicals can preferably be selected from the above group of substituents for linear alkyl groups without restricting the invention to these substituents. Particularly preferred substituents for aryl groups of the aralkyl radicals are the substituents —Cl, —Br, methyl, ethyl, n-propyl, i-propyl, n-butyl, butyl, sec-butyl or tert-butyl, methoxy, ethoxy, n-propoxy, i-propoxy; n-butoxy, i-butoxy, sec-butoxy, tert-butoxy, —NH₂, —NH(CH₃), —N(CH₃)₂, —NH(C₂H₅) and —N(C₂H₅)₂, carbonyl and carboxyl. One or more such substituents, which can be the same or different, can be bonded to an aryl group of an aralkyl radical according to the present invention. The substituted position(s) on the aryl ring (system) can be selected as desired.

In preferred embodiments of the invention heteroarylalkyl radicals are radicals, in which the heteroaryl radical of the heteroarylalkyl radicals is substituted according to the invention and the alkylene group is straight-chain or branched and has 1 to 6 carbon atoms. The ring structure of such a heteroaryl radical can be a five-, six- or seven-membered ring structure with a ring or a structure formed from two or more rings bonded to one another (annellated), wherein the annellated rings can have the same or a different number of ring members. The heteroatom(s) can be present alone in one or also in several of the rings of the ring system. The heteroaryl radicals of the heteroarylalkyl radicals preferably comprise one or two rings. In the case of heteroarylalkyl systems composed of a plurality of rings annellated to one another, benzo-condensed rings are particularly preferred, i.e. ring systems in which at least one of the rings is an aromatic carbocyclic six-membered ring. Particularly preferred heteroarylalkyl radicals are selected from furanylmethyl and-ethyl, thiophenylmethyl and -ethyl, pyridylmethyl and -ethyl, indolylmethyl and -ethyl, cumaronylmethyl and -ethyl, thionaphthenylmethyl and -ethyl, quinolinyl-(benzopyridyl-)methyl and -ethyl, quinazolinyl-(benzopyrimidinyl-)methyl and -ethyl and quinoxylinyl-(benzopyrazinyl-)methyl and -ethyl.

Possible substituents on these heteroaryl groups of the heteroarylalkyl radicals can preferably be selected from the above group of substituents for linear alkyl groups without restricting the invention to these substituents. Particularly preferred substituents for heteroaryl groups are the substituents —Cl, —Br, methyl, ethyl, n-propyl, i-propyl, n-butyl, i-butyl, sec-butyl or tert-butyl, methoxy, ethoxy, n-propoxy, i-propoxy; n-butoxy, i-butoxy, sec-butoxy, tert-butoxy, —NH₂, —NH(CH₃), —N(CH₃)₂, —NH(C₂H₅) and —N(C₂H₅)₂, carbonyl and carboxyl. One or more such substituents, which can be the same or different, can be bonded to a heteroarylalkyl radical according to the present invention. The substituted position(s) on the heteroaryl ring (system) can be selected as desired.

In preferred embodiments of the invention, heterocycloalkyl radicals are cycloalkyl radicals in accordance with the above general and specific definition, which contain one or more heteroatoms, which is/are selected from —O—, —S— and —NRₓ—, wherein Rₓ stands for hydrogen or an alkyl radical (as defined above) with 1 to 6 carbon atoms, and the alkyl(ene) groups of the heterocycloalkyl radicals are straight-chain or branched and have 1 to 6 carbon atoms. In the cases where several heteroatoms are bound into the ring(s), these can be the same or different. One heteroatom is preferably bound into the ring. In further embodiments of the invention, preferred examples of cycloalkyl radicals containing heteroatoms, also referred to as heterocycloalkyl radicals, are the radicals tetrahydrofuranyl, pyrrolidinyl, pyrazolidinyl, imidazolidinyl, piperidinyl, piperazinyl and morpholinyl.

Possible substituents on these heterocycloalkyl radicals can preferably be selected from the above group of substituents for linear alkyl groups without restricting the invention to these substituents. Particularly preferred substituents for heteroaryl groups are the substituents —Cl, —Br, methyl, ethyl, n-propyl, i-propyl, n-butyl, i-butyl, sec-butyl or tert-butyl, methoxy, ethoxy, n-propoxy, i-propoxy; n-butoxy, i-butoxy, sec-butoxy, tert-butoxy, —NH₂, —NH(CH₃), —N(CH₃)₂, —NH(C₂H₅) and —N(C₂H₅)₂, carbonyl and carboxyl. One or more such substituents, which can be the same or different, can be bonded to a heterocycloalkyl radical according to the present invention. The substituted position(s) on the heterocycloalkyl ring (system) can be selected as desired.

In the present description and the patent claims, the terms “arylamidoalkyl radical” and “heteroarylamidoalkyl radical” mean alkyl radicals (or—more precisely—alkylene radicals) in the sense of the above general and specific definition, which are substituted at one of their bonds with an arylamido radical or heteroarylamido radical with the general formula Ar—NRₓ—C(═O)— or the general formula Ar—C(═O)—NRₓ—, wherein Rₓ stands for hydrogen or an alkyl radical with 1 to 6 carbon atoms and Ar stands for any desired aryl radical or heteroaryl radical in accordance with the above general or specific definition. These aryl or heteroaryl radicals can be unsubstituted or substituted. Preferred examples of an arylamidoalkyl radical—without restricting the invention in this regard—are 2-, 3- or 4-benzoic acid amido-n-butyl radicals or 2-nitro-3-, -4-, -5- or -6-benzoic acid amido-n-butyl radicals; preferred, but not restrictive examples of heteroarylamidoalkyl radicals are 2-, 4-, 5- or 6-pyridine-3-carboxylic acid-amido-n-butyl radicals.

Possible substituents on these arylamidoalkyl radicals and heteroarylamidoalkyl radicals can preferably be selected from the above group of substituents for linear alkyl groups without restricting the invention to these substituents. Particularly preferred substituents for aryl groups or heteroaryl groups of the arylamidoalkyl radicals and heteroarylamidoalkyl radicals are the substituents —Cl, —Br, methyl, ethyl, n-propyl, i-propyl, n-butyl, i-butyl, sec-butyl or tert-butyl, methoxy, ethoxy, n-propoxy, i-propoxy; n-butoxy, i-butoxy, sec-butoxy, tert-butoxy, —NH₂, —NH(CH₃), —N(CH₃)₂, —NH(C₂H₅) and —N(C₂H₅)₂, carbonyl and carboxyl. One or more such substituents, which can be the same or different, can be bonded to an aryl or heteroaryl group of the arylamidoalkyl radicals or heteroarylamidoalkyl radicals according to the present invention. The substituted position(s) on the aromatic ring (system) can be selected as desired.

A comparable definition to that in the case of aralkyl radicals, heteroarylalkyl radicals, heterocycloalkyl radicals, arylamidoalkyl radicals and heteroarylamidoalkyl radicals applies within the framework of the present description and the patent claims with respect to the definition of the terms “aralkylene radical”, “heteroarylalkylene radical”, “heterocycloalkylene radical”, “arylamidoalkylene radical” and “heteroarylamidoalkylene radical”. These are respectively understood to relate to divalent radicals, the fundamental structure and selection and substituent(s) of which are comparable to the above details for the definition of the “aralkyl radical”, “heteroarylalkyl radical”, “heterocycloalkyl radical”, “arylamidoalkyl radical” and “heteroarylamidoalkyl radical”, except that in each case this is a divalent radical that can be bonded to any two carbon atoms of the ring or ring system or the alkylene group or also to a nitrogen atom of the heteroaryl or heterocyclyl ring system.

In the general formulae (1) and (2) the radical D stands for —S—S— or —Se—Se—. These two S or Se atoms form a bridge between two parts of the molecule of the compounds of the general formulae (1) and (2), which can be split under natural, in particular reducing conditions. In this case, two molecule parts can be released, which develop an inhibitory effect with respect to dipeptidylpeptidase IV (DP IV) and peptidases with analogous enzymatic effect and also with respect to alanyl-aminopeptidase N (APN) and peptidases with analogous enzymatic effect.

In the above general formula (2), E stands for the group —CH₂—C*H (NH₂)—R⁹, wherein R⁹ stands for an unsubstituted or substituted, unbranched or branched alkyl radical, cycloalkyl radical, aralkyl radical, heterocycloalkyl radical, heteroarylalkyl radical, arylamidoalkyl radical, heteroarylamidoalkyl radical, containing or not containing O, N or S, unsubstituted or mono- or polysubstituted aryl radical or heteroaryl radical with one or more five-, six- or seven-membered ring(s). With respect to the examples preferred or usable according to the invention for alkyl radicals, cycloalkyl radicals, aralkyl radicals, heterocycloalkyl radicals, heteroarylalkyl radicals, arylamidoalkyl radicals, heteroarylamidoalkyl radicals, unsubstituted or mono- or polysubstituted aryl radicals or heteroaryl radicals with one or more five-, six- or seven-membered ring(s) as well as the preferred substituents conceivable for these radicals, reference can be made to the above definition of the corresponding radicals and their preferred embodiments. These definitions are also applicable in the same way to the radicals of the general formula (2) which E stands for.

In the above formula for E * represents a chiral carbon atom on the carbon atom substituted with the amino group. In further preferred embodiments of the invention, such compounds of the general formula (2) represent prodrugs to particularly effective inhibitors, in which the chiral carbon atom designated by * has an S- or L-configuration.

It is particularly preferred according to the invention if E stands for substituted 2-aminoalkylene radicals, e.g. a 2-amino-3-phenylpropyl radical, or for 2-aminoalkylene radicals that are unsubstituted or substituted by heteroatoms such as —S—, —S(═O)—, —N— or —O—, e.g. a 2-amino-4-methylpentyl radical, a 2-amino-4-methylthiobutyl radical or a 2-amino-4-methyl-sulphoxybutyl radical.

In further preferred embodiments of the invention, the radicals B and/or B′ in the general formulae (1) and (2) stand for a radical R′, which stands for a straight-chain or branched alkylene radical with 1 to 6 carbon atoms. Particularly preferred compounds of the general formulae (1) and (2) comprise B and/or B′ radicals in the form of one or more of the groups selected from —CH₂-(methylene), —CH₂—CH₂-(ethylene) or (H₃C)₂-C<(2,2-propylene).

In alternative, likewise further preferred embodiments, B and/or B′ stand for a radical —(CH₂)ₙ—R²—R³—R⁴—, wherein n stands for a whole number from 1 to 5; R² stands for —NH— or —NH—C(═NH)—NH— when R³ stands for O═C< or —SO₂—, or wherein R² stands for O═C< when R³ stands for —NH—; R⁴ stands for an unsubstituted or substituted, unbranched or branched alkylene radical, cycloalkylene radical, aralkylene radical, heterocycloalkylene radical, heteroarylalkylene radical, containing or not containing O, N or S, unsubstituted or mono- or polysubstituted arylene radical or heteroarylene radical with one or more five-, six- or seven-membered ring(s). It is further preferred if n stands for 1 to 5, so that preferred examples of the aforementioned radical comprise a methylene group, ethylene group, propylene group, butylene group and pentylene group; R² and R³ preferably together form an amido group —C(═O)—NH— or —NH—C (═O)—. Those compounds of the general formulae (1) and (2) are further preferred that have B and/or B′ radicals, wherein B stands for the aforementioned formula and R⁴ represents an amino-substituted alkylene radical, e.g. an aminoethylene radical, or an unsubstituted or substituted (e.g. with a nitro group) phenylene radical or an unsubstituted or substituted pyridyl-2,5-ene radical.

In alternative, likewise further preferred embodiments, B and/or B′ stand for a radical of the formula —R⁷—R⁸—, wherein R⁷ stands for a mono- or polysubstituted benzylene radical and R⁸ stands for a single bond or an unsubstituted or substituted, unbranched or branched alkylene radical, cycloalkylene radical, aralkylene radical, heterocycloalkylene radical or heteroarylalkylene radical, containing or not containing O, N or S, which can preferably have one or more amino groups, carbonyl groups or carboxyl groups as functional groups, or an unsubstituted or mono- or polysubstituted arylene radical or heteroarylene radical with one or more five-, six- or seven-membered ring(s). The preceding general or specific definitions of the respective radicals and substituents can be referred to with respect to the definition of the aforementioned radicals and their conceivable substituents according to the invention.

Further preferred according to the invention are compounds of the general formulae (1) and (2), in which B and B′ can be the same or different and stand for a radical —(CH₂)ₙ—R²—R³—R⁴—, wherein R² stands for —NH— or —NH—C(═NH)—NH— when R³ stands for O=C< or —SO₂—, or wherein R² stands for O═C< when R³ stands for —NH—; and wherein R⁴ stands for
—CH(COOH)—R¹, wherein R¹ has the meaning specified above when R² stands for O═C<

and R³ stands for —NH—; or

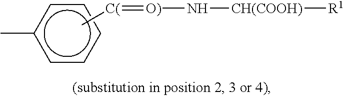

wherein R¹ has the meaning specified above when R² stands for O═C< and R³ stands for —NH—; or
—CH(NHR⁵)—R¹— when R² stands for —NH— or —NH—C(═NH)—NH— and R³ stands for O═C<, wherein R⁵ stands for H or an acyl radical, preferably for a benzyloxycarbonyl radical, a fluoren-9-ylmethoxycarbonyl radical, a tert-butyloxycarbonyl radical or a benzoyl radical; or

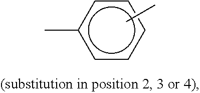

wherein R⁴ stands for phenylene and R² stands for —NH— or —NH—C(═NH)—NH— when R³ stands for O═C< or —SO₂—, or wherein R² stands for O═C< when R³ stands for —NH—; or

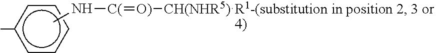

wherein R⁵ stands for H or an acyl radical, preferably for a benzyloxycarbonyl radical, a fluoren-9-ylmethoxycarbonyl radical or a benzoyl radical, and R² stands for —NH— or —NH—C(═NH)—NH— when R³ stands for O═C< or —SO₂—, or wherein R² stands for O═C< when R³ stands for —NH—; or

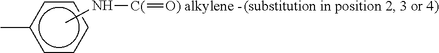

wherein alkylene stands for an unbranched or branched alkylene radical with 1 to 6 carbon atoms and R² stands for —NH— or —NH—C(═NH)—NH— when R³ stands for O═C< or —SO₂—, or wherein R² stands for O═C< when R³ stands for —NH—; or

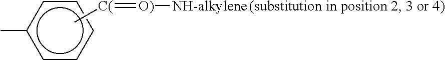

wherein alkylene stands for an unbranched or branched alkylene radical with 1 to 6 carbon atoms and R² stands for —NH— or —NH—C(═NH)—NH— when R³ stands for O═C< or —SO₂—, or wherein R² stands for O═C< when R³ stands for —NH—; or

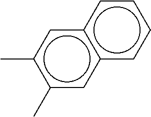

wherein R² stands for —NH— or —NH—C(═NH)—NH— when R³ stands for O═C< or —SO₂—, or wherein R² stands for O═C< when R³ stands for —NH—; or

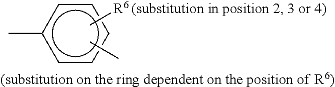

wherein R⁶ stands for H, NO₂, CN, halogen or an acyl radical and R² stands for —NH— or —NH—C(═NH)—NH— when R³ stands for O═C< or —SO₂—, or wherein R² stands for O═C< when R³ stands for —NH—; or

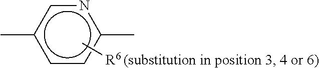

wherein R⁶ stands for H, NO₂, CN, halogen or an acyl radical and R² stands for —NH— or —NH—C(═NH)—NH— when R³ stands for O═C< or —SO₂—, or wherein R² stands for O═C< when R³ stands for —NH—; or

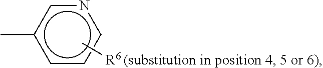

wherein R⁶ stands for H, NO₂, CN, halogen or an acyl radical and R² stands for —NH— or —NH—C(═NH)—NH— when R³ stands for O═C< or —SO₂—, or wherein R² stands for O═C< when R³ stands for —NH—l ; or

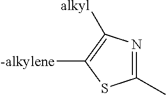

wherein R² stands for —NH— or —NH—C(═NH)—NH— when R³ stands for O═C< or —SO₂—, or wherein R² stands for O═C< when R³ stands for —NH—.

Alternatively, further preferred compounds of the general formulae (1) and (2) according to the invention are those in which B and B′ can be the same or different and stand for a radical —R⁷—R⁸—, wherein R⁷ and R⁸ in combination stand for a radical

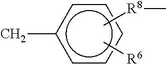

(in which R⁷ stands for the above radical without R⁸ and the position of R⁶ is dependent on the position of R⁸), wherein R⁸ and R⁶ have the above-specified meanings, i.e. wherein R⁶ stands for H, NO₂, CN, halogen or an acyl radical and wherein R⁸ stands for a single bond or for an unsubstituted or substituted, unbranched or branched alkylene radical, cycloalkylene radical, aralkylene radical, heterocycloalkylene radical or heteroarylalkylene radical, containing or not containing O, N or S, which can preferably have one or more amino groups, carbonyl groups or carboxyl groups as functional groups, or for an unsubstituted or mono- or polysubstituted arylene radical or heteroarylene radical with one or more five-, six- or seven-membered ring(s).

Even further preferred compounds of the general formulae (1) and (2) are those in which B and 13¹ are the same or different and independently of one another stand for a radical —R⁷—R⁸—, wherein R⁷ stands for a mono- or polysubstituted benzylene radical of the above formula (without R⁸) and R⁸ stands for NH— or —C₁- to C₆-alkylene-NH— in combination with
—C(═O)—C₁- to C₆-alkylene- or
—C(═O)-arylene- or
—SO₂13 C₁- to C₆-alkylene- or
—SO₂-arylene- or

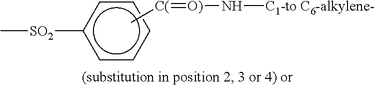

—C(═O)—CH(NHR⁵)—R¹, wherein R¹ and R⁵ have the above-specified meanings; or
O═C< in combination with
—NH—C₁- to C₆-alkylene- or
—NH-arylene- or
—NH—CH(COOH)—R¹—, wherein R¹ has the above-specified meanings; or
—O—C₁- to C₆-alkylene- or
—O-arylene- or
—O—C₁- to C₆-alkylene-NH—C(═O)—CH(NH₂)—R¹—, wherein R¹ has the above-specific meanings, or
—O—C₁- to C₆-alkylene-C(═O)—NH—CH(COOH)—R¹—, wherein R¹ has the above-specified meanings.

According to the invention, the compounds of the general formulae (1) and/or (2) are present in the form of neutral molecules and as such have a use according to the invention in the activation of Treg cells. Alternatively, the compounds of the general formulae (1) and/or (2) can also be present in the form of their acid addition salts with inorganic and/or organic acids. Such acid addition salts are formed because of the presence of basic sites (mostly of basic nitrogen atoms) in the molecule by attachment of one or more molecules of H-acid compounds (Brönsted acids), preferably a molecule of an H-acid compound, and assure an improved solubility of the molecules in polar media such as water, for example. The last-mentioned property is particularly important for those compounds that develop pharmacological effects.

In preferred embodiments of the invention, the acid addition salts are pharmaceutically acceptable acids and are advantageously (but without restriction for the present invention) selected from the group comprising hydrochlorides, trifluoroacetates, tartrates, succinates, formiates and/or citrates of the compounds of the general formulae (1) and (2).

Particularly preferred and advantageously usable compounds of general formula (1) are characterised by general formula (1a)

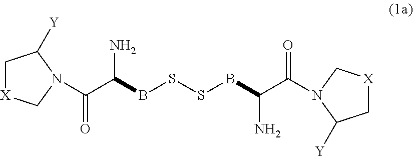

wherein X, Y and B have the above-specified meanings. Acid addition salts of the compound of general formula (la), preferably their acid addition salts with pharmaceutically acceptable inorganic and/or organic acids, in particular with acids from the aforementioned group, most particularly preferred hydrochlorides, trifluoroacetates, tartrates, succinates, formiates and/or citrates of the compounds of the general formula (la), are usable with particular advantage in the method according to the invention.

Most particularly preferred compounds of the general formula (1a) are shown in the following Table 1 without the invention being restricted to these compounds.

TABLE 1
Examples of Compounds of General Formula A—B—D—B′—A′ (1)

| | | | | Empirical |
|---|---|---|---|---|
| No. | B | X | Y | Formula |
| I | —CH₂— | —CH₂— | H | C₁₄H₂₆N₄O₂S₂ |
| II | —CH₂— | S | H | C₁₂H₂₂N₄O₂S₄ |
| III | —CH₂— | —CH₂— | CN | C₁₆H₂₄N₆O₂S₂ |
| IV | 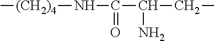 | S | H | C₂₄H₄₆N₈O₄S₄ |
| V | 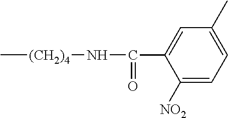 | S | H | C₃₂H₄₂N₈O₈S₄ |
| VI | 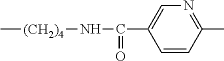 | S | H | C₃₀H₄₂N₈O₄S₄ |

and their acid addition salts, preferably their acid addition salts with pharmaceutically acceptable inorganic and/or organic acids, preferably pharmaceutically acceptable acids from the aforementioned group, most particularly preferred hydrochlorides, trifluoroacetates, tartrates, succinates, formiates and/or citrates of the compounds of the general formula (1a).

Particularly preferred and advantageously usable compounds of general formula (2) are characterised by general formula (2a)

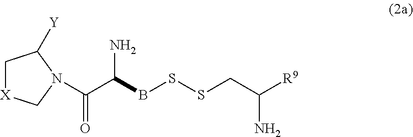

wherein X, Y, R⁹ and B have the above-specified meanings, and their acid addition salts, preferably their acid addition salts with pharmaceutically acceptable inorganic and/or organic acids, preferably pharmaceutically acceptable acids from the aforementioned group, particularly preferred hydrochlorides, trifluoroacetates, tartrates, succinates, formiates and/or citrates of the compounds of the general formula (2a).

Most particularly preferred compounds of general formula (2a) are shown in the following Table 2 without the invention being restricted to these compounds.

TABLE 2
Examples of Compounds of General Formula A—B—D—E (2)

| No. | B | R⁹ | X | Y | Empirical Formula |
|---|---|---|---|---|---|
| VII | —CH₂— | 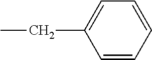 | S | H | C₁₅H₂₃N₃OS₃ |
| VIII | 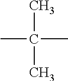 | 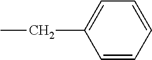 | S | H | C₁₇H₂₇N₃OS₃ |
| IX | 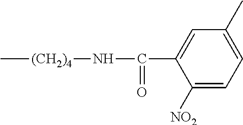 | 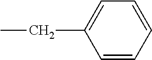 | S | H | C₂₅H₃₃N₅O₄S₃ |
| X | 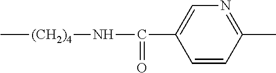 | 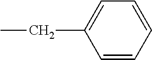 | S | H | C₂₄H₃₃N₅O₂S₃ |
| XI | 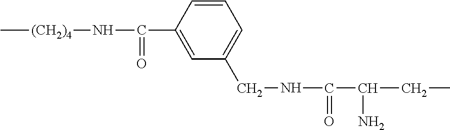 | 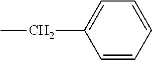 | S | H | C₂₉H₄₂N₆O₃S₃ |
| XII | 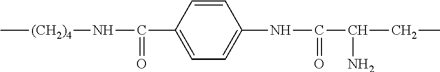 | 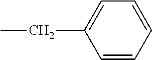 | S | H | C₂₆H₄₀N₆O₃S₃ |
| XIII | 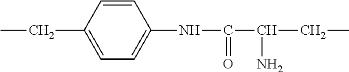 | 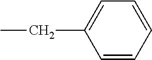 | S | H | C₂₄H₃₃N₅O₂S₃ |

and their acid addition salts, preferably their acid addition salts with pharmaceutically acceptable inorganic and/or organic acids, particularly preferred hydrochlorides, trifluoroacetates, tartrates, succinates, formiates and/or citrates of the compounds of general formula (2a).

The inhibitors are used in a concentration that corresponds to the IC₅₀ inhibition value or lies above this. The inhibitor concentration lies in the nanomolecular to micromolecular range and can be determined by a person skilled in the art in a few easily conducted standard experiments without any difficulty. The cultivation of Treg cells with the one or the plurality of inhibitors according to the above detailed description is preferably conducted at 37° C., more preferred in an atmosphere of steam-saturated air with a CO₂ content of 5%, for example. The concentration of Treg cells is adapted to the total volume and particularly preferred lies at 1 to 5 million cells per ml.

In a further, likewise preferred embodiment of the method according to the invention, in addition to the one or the plurality of inhibitor(s) of alanyl-aminopeptidase (aminopeptidase N; APN) and/or in addition to the one or the plurality of inhibitor(s) of peptidases with the same substrate specificity, peptide fragments of pathogenic autoantigens or synthetic analogues and/or specific antigenic components of pathogenic microorganisms are used. One type of peptide fragments can be used or several types of peptide fragments can be used. In addition, it is possible that one type of specific antigenic components of pathogenic microorganisms is used, or that several types of specific antigenic components of pathogenic microorganisms are used. According to the invention, combinations of one or more of the said components can also be used. Surprisingly, a particularly favourable activation of regulatory T-cells (Treg cells) can be achieved with this combination of inhibitor(s) and further component(s).

In further preferred embodiments of the method according to the invention, MBP (myelin basic protein), MOG (myelin oligodendrocyte glycoprotein), MAG (myelin associated glycoprotein) and/or PLP (proteolipid protein) is/are used. According to another, likewise further preferred embodiment of the method, coat proteins or membrane glycolipid complexes are used as specific antigenic components of pathogenic microorganisms. Combinations of the special components can also be used.

According to the invention, regulatory T-cells (Treg cells) are brought into contact with the medium or media described in detail above in the manner known to a skilled person in this technical field. Given purely by way of example (and without restriction to the present invention), it is stated that the step of bringing into contact or incubating regulatory T-cells (Treg cells) with one or more inhibitors of alanyl-amidopeptidase (aminopeptidase N; APN) and/or with one or more inhibitors of peptidases with the same substrate specificity is conducted in customary, preferably static or horizontally moved or vertically moved or rotationally moved cell culture vessels. More preferred, these can be culture dishes, culture plates, cell culture reactors, cell culture flasks, cell culture bags, dual- or multi-chambered systems suitable for cell cultures or hollow fibre reactors or any other vessels known to the skilled person that are generally used for cultivating cells. It is particularly preferred if the cultivation is conducted in cell culture vessels, which have a possibly reaction-promoting surface coating and/or matrix substitutes on a part or on the whole of these surface directed towards the culture. The cell culture is preferably conducted in the presence of 5% CO₂ in steam-saturated air at 37° C.

In the last step of the method according to the invention, the regulatory T-cells (Treg cells) activated in the aforementioned manner are returned to at least one human or animal body in a suitable medium. The Treg cells are regularly conveyed to a human or animal body that requires these activated Treg cells to regulate an immune problem. In any case, the medium is a medium that is pharmaceutically acceptable for the recipient and in further preferred embodiments of the invention can be selected from preferably fluid, further preferred liquid media from the group of physiologically acceptable solutions, particularly preferred aqueous solutions that, if necessary, can contain further useful or even expedient components for the planned purpose of use.

It is particularly preferred if the activated Treg cells are conveyed to the organism of the (human or animal) donor of the body fluid, from which the Treg cells were isolated. The return can occur in any manner conceivable to the skilled person that fulfils the desired purpose, i.e. conveys the activated Treg cells to the recipient organism (whether human or animal) again. Particularly preferred ways of return are forms of application selected from the group comprising intravenous application, intra-arterial application, intracavitary application, intrathecal application and intradermal application. An intravenous application is preferably used to particular advantage if the intention is to infuse the activated Treg cells into the recipient again, since this enables a direct insertion of the Treg cells into the peripheral system and thus into the blood circulation, where the Treg cells also act naturally.

The infused quantity of T-cells (Treg cells) is heavily dependent on their concentration in the medium used for the return infusion, the constitution of the recipient (human or animal), the clinical picture or the immune status and on other factors known to or easily determined by a skilled person.

The invention also relates to activated regulatory T-cells (Treg cells) such as those obtainable using the method described in detail above with one or more inhibitors of alanyl-aminopeptidase and/or with one or more inhibitors of peptidases with the same substrate specificity. Such activated Treg cells were not known until today and possess a surprisingly high suppressive effect compared with Treg cells that have been activated in the conventional manner. In particular, the regulatory T-cells (Treg cells) activated using the method according to the invention can be employed to generate tolerance towards autoantigens (antigens produced within the organism) and alloantigens (antigens introduced by external factors) in the human and animal body and to overcome an excessive immune response in the human and animal body, since they suppress the immune response of the body to a surprisingly high degree.

The invention also relates to preparations of any type comprising or containing activated regulatory T-cells (Treg cells) that can be activated using the method described in detail above with one or more inhibitors of alanyl-aminopeptidase and/or with one or more inhibitors of peptidases with the same substrate specificity. Besides the activated regulatory T-cells and the support medium or solvent suitable for administration, such preparations can possibly additionally contain one or more supports, auxiliary substances and/or adjuvants. These can include—as known to the skilled person—one or more components known to the skilled person as support, auxiliary substance and/or adjuvant used singly or in combination.

The invention additionally relates to the use of activated regulatory T-cells (Treg cells) or also the use of preparations comprising activated regulatory T-cells (Treg cells) for the prevention, alleviation or therapy of numerous diseases and conditions related to or associated with an imbalanced immune reaction of the human or animal body. It is unimportant in the use according to the invention what inhibitor or inhibitors have been used to activate the Treg cells.

The activated regulatory T-cells (Treg cells) as well as preparations containing these have proved particularly beneficial in the prevention, alleviation or therapy of transplant rejections.

The invention also relates to the use of activated regulatory T-cells (Treg cells) or also the use of preparations comprising activated regulatory T-cells (Treg cells) for the prevention, alleviation or therapy of diseases with an excessive immune response and inflammatory genesis including arteriosclerosis, neuronal diseases, brain damage, skin diseases such as e.g. psoriasis, acne, keloids and other hyperproliferative conditions as well as sepsis and type II diabetes.

The invention also relates to the use of activated regulatory T-cells (Treg cells) or also the use of preparations comprising activated regulatory T-cells (Treg cells) for the production of a medication or a cosmetic preparation for the prevention, alleviation or therapy of diseases with an excessive immune response and inflammatory genesis including arteriosclerosis, neuronal diseases, brain damage, skin diseases such as e.g. psoriasis, acne, keloids and other hyperproliferative conditions, fibroses, tumour diseases and virus-related illnesses, as well as sepsis and type II diabetes.

In preferred embodiments of the invention, the activated regulatory T-cells (Treg cells) or the preparations containing these are used for the prophylaxis and therapy of diseases such as e.g. multiple sclerosis, Crohn's disease, ulcerative colitis, and other autoimmune disorders as well as inflammatory diseases, bronchial asthma and other allergy disorders, skin and mucous membrane diseases, e.g. psoriasis, acne as well as dermatological diseases with hyperproliferation and altered differentiation conditions of fibroblasts, benign fibrosing and sclerosing skin diseases and malignant fibroblastic hyperproliferation conditions, acute neuronal diseases such as e.g. ischaemia-related brain damage conditions after an ischaemic or haemorrhagic stroke, cranio-cerebral trauma, cardiac arrest, myocardial infarction or as a result of heart surgery, chronic neuronal diseases, e.g. Alzheimer's disease, Pick's disease, progressive supranuclear palsy, corticobasal degeneration, frontotemporal dementia, Parkinson's disease, in particular Parkinsonism linked to chromosome 17, Huntington's disease, prion-related disease conditions and amyotrophic lateral sclerosis, atherosclerosis, arterial inflammation, stent restenosis, chronic obstructive pulmonary diseases (COPD), tumours, formation of metastases, prostate cancer, severe acute respiratory syndrome (SARS), and of sepsis and sepsis-like conditions, as well as type II diabetes.

In a further preferred embodiment of the invention, the activated regulatory T-cells (Treg cells) or the preparations containing these are used for the prophylaxis and therapy for the rejection of transplanted tissues and cells. An example of such an application can be the use of regulatory T-cells (Treg cells) or the use of a preparation comprising Treg cells in allogenic or xenogenic transplanted organs, tissues and cells such as in kidney, heart, liver, pancreas, skin or stem cell transplantation as well as graft-versus-host reactions.

In a further preferred embodiment of the invention, the activated regulatory T-cells (Treg cells) or the preparations containing these are used for the prophylaxis and therapy for rejection or inflammatory reactions at or as a result of medical devices implanted into the organism. These can be, for example, stents, joint implants (knee joint implants, hip joint implants), bone implants, pacemakers or other implants.

In a further preferred embodiment of the invention, the activated regulatory T-cells (Treg cells) or the preparations containing these are used, so that the Treg cells or composition(s) containing these are applied to the device or devices in the form of a coating or wetting layer, or at least the regulatory T-cells (Treg cells) or the compositions containing these are integrally mixed with the material of the device/devices. Of course, it is also possible in this case to administer activated Treg cells that have been produced using the method according to the invention, or compositions containing these, locally or systemically—possibly at intervals in time or in parallel.

In the same way as described above—and for the comparable purposes or for prophylaxis and therapy for the diseases and conditions specified above by way of example, but not definitively—the regulatory T-cells (Treg cells) in general and the pharmaceutical and cosmetic compositions containing them can be used alone or in combinations of several for the production of medications for the treatment of the abovementioned illnesses or conditions. These can comprise the activated regulatory T-cells (Treg cells) in the quantities given by way of example below, possibly together with support, auxiliary substances and/or additives known per se.

The invention is explained in more detail below by means of examples of application. However, it is to be understood in respect of the above detailed disclosure that the invention is not restricted to the following examples. The following examples represent the currently preferred best embodiments.

### EXAMPLES

### Example 1

### Activation of Human Regulatory T-cells in the Presence of Actinonin as Inhibitor of Aminopeptidase N

Mononuclear cells were obtained from the peripheral blood of healthy donors by means of density-gradient centrifugation. The isolation of regulatory T-cells occurred by means of a two-stage magnetic separation:

In a first step CD4⁺ T-cells were recovered by depletion of all CD4-negative cells [CD4 separation kit, Miltenyi Biotech, Bergisch-Gladbach, Germany]. The purity achieved regularly amounted to >95% CD4⁺ T-cells.

In a second step CD4⁺CD25⁺ regulatory T-cells were in turn isolated from this population by magnetic column separation using CD25 marking [anti-CD25 MicroBeads, Miltenyi Biotech].

The CD4⁺CD25⁻ fraction served as effector cell control.

The functional capacity of the regulatory T-cells was tested in a special co-culture. For this, 20 000 effector cells (CD4⁺CD25⁻) and regulatory T-cells (CD4⁺CD25⁺) in total were respectively cultivated in different quantitative ratios to one another over a period of 120 hours in microtest plates. A solid phase-bound anti-CD3 antibody [UCHT1, 0.25 μg/well] was used as activator of the T-cell stimulation.

The degree of proliferation of the cultivated cells was analysed on the basis of the DNA synthesis rate by means of tritium thymidine inclusion over 24 hours [n=5].

The diagram (FIG. 1) shows the induction of the suppressive phenotype of regulatory T-cells (Treg cells) in the quantitative relations of 50% (experimentally relevant), 20% (experimentally relevant) and 10% (physiologically relevant) Treg components in the presence and absence of the APN inhibitor, actinonin.

While with a ratio of 1:1 of effector cells to Treg cells no reliable effect of the inhibitor was evident because of the strong suppressive capacity of the Treg cells, a significant intensification of the suppressive capacity of the Treg cells became clear in particular in the physiologically relevant quantitative range of 10:1 (p<0.05).

### Example 2

Activation of Human Regulatory T-cells in the Presence of PAQ22 as Inhibitor of Cytosolic Aminopeptidase (cAAP)

Mononuclear cells were obtained from the peripheral blood of healthy donors by means of density-gradient centrifugation. The isolation of regulatory T-cells occurred by means of a two-stage magnetic separation:

In a first step CD4⁺ T-cells were recovered by depletion of all CD4-negative cells [CD4 separation kit, Miltenyi Biotech, Bergisch-Gladbach, Germany]. The purity achieved regularly amounted to >95% CD4⁺ T-cells.

In a second step CD4⁺CD25⁺ regulatory T-cells were in turn isolated from this population by magnetic column separation using CD25 marking [anti-CD25 MicroBeads, Miltenyi Biotech].

The CD4⁺CD25⁻ fraction served as effector cell control.

The functional capacity of the regulatory T-cells was tested in a special co-culture. For this, 20 000 effector cells (CD4⁺CD25⁻) and regulatory T-cells (CD4⁺CD25⁺) in total were respectively cultivated in different quantitative ratios to one another over a period of 120 hours in microtest plates. A solid phase-bound anti-CD3 antibody [UCHT1, 0.25 μg/well] was used as activator of the T-cell stimulation.

The degree of proliferation of the cultivated cells was analysed on the basis of the DNA synthesis rate by means of tritium thymidine inclusion over 24 hours [n=3].

The diagram (FIG. 2) shows the induction of the suppressive phenotype of regulatory T-cells (Treg cells) in the physiologically relevant quantitative relations of 10% and 5% Treg cell components in the presence and absence of the selective inhibitor of cytosolic aminopeptidase (cAAP), PAQ22.

PAQ22 induced the suppressive capacity of the regulatory T-cells after 5 days of co-culture, depending on concentration.

### Example 3

### Activation of Human Regulatory T-cells in the Presence of IP10.C8 as Dual Inhibitor of Alanyl Aminopeptidase (APN) and Dipeptidylpeptidase IV (DPIV)

Mononuclear cells were obtained from the peripheral blood of healthy donors by means of density-gradient centrifugation. A T-cell separation followed using nylon pad adherence.

The isolation of regulatory T-cells occurred by means of a two-stage magnetic separation:

In a first step CD4⁺ T-cells were recovered by depletion of all CD4-negative cells [CD4 separation kit, Miltenyi Biotech, Bergisch-Gladbach, Germany]. The purity achieved regularly amounted to >95% CD4⁺ T-cells.

In a second step CD4⁺CD25⁺ regulatory T-cells were in turn isolated from this population by magnetic column separation using CD25 marking [anti-CD25 MicroBeads, Miltenyi Biotech].

The CD4⁺CD25⁻ fraction served as effector cell control.

The functional capacity of the regulatory T-cells was tested in a special co-culture. For this, 20 000 effector cells (CD4⁺CD25⁻) and regulatory T-cells (CD4⁺CD25⁺) in total were respectively cultivated in different quantitative ratios to one another over a period of 120 hours in microtest plates. A solid phase-bound anti-CD3 antibody [UCHT1, 0.25 μg/well] was used as activator of the T-cell stimulation.

The degree of proliferation of the cultivated cells was analysed on the basis of the DNA synthesis rate by means of tritium thymidine inclusion over 24 hours [n=10].

The diagram (FIG. 3) shows the induction of the suppressive phenotype of regulatory T-cells (Treg cells) in the quantitative relations of 50% (experimentally relevant), 20% (experimentally relevant) and 10% (physiologically relevant) Treg cell components in the presence and absence of the dual inhibitor of aminopeptidase N and dipeptidylpeptidase IV, IP10.C8.

While with a ratio of 1:1 of effector cells to Treg cells no reliable effect of the inhibitor was evident because of the strong suppressive capacity of the Treg cells, a significant intensification of the suppressive capacity of the Treg cells became clear in particular in the physiologically relevant quantitative range of 10:1 (p<0.01).

### Example 4

### Activation of Murine Regulatory T-cells in the Presence of Phebestin as Inhibitor of Alanyl-Aminopeptidase (APN)

Mononuclear cells (MNC) were obtained from the peripheral blood of healthy mice by means of density-gradient centrifugation. The isolation of regulatory T-cells occurred using CD25 marking [anti-CD25 MicroBeads, Miltenyi Biotech]. The CD25⁻ MNC fraction served as effector cell control.

The functional capacity of the regulatory T-cells was tested in a special co-culture. For this, 20 000 effector cells (MNC-CD25⁻) and regulatory T-cells (CD4⁺CD25⁺) in total were respectively cultivated in different quantitative ratios to one another over a period of 120 hours in microtest plates. The T-cell stimulation occurred by adding anti-CD3/anti-CD28 [1 μg/ml].

The degree of proliferation of the cultivated cells was analysed on the basis of the DNA synthesis rate by means of tritium thymidine inclusion over 24 hours [n=4].

The diagram (FIG. 4) shows the induction of the suppressive phenotype of regulatory T-cells (Treg cells) in the presence and absence of the APN inhibitor, phebestin.

After 5 days of co-culture phebestin [1.0 μM] induced the suppressive capacity of the regulatory T-cells to a significant degree (* p<0.01, ^{#}p<0.05).

### Example 5

Effect of Regulatory T-cells (Treg Cells) Activated Ex-situ with an Inhibitor of APN (Phebestin) in the Colitis Model in Mice

Colitis was triggered in Balb-c mice by the oral application of 3% dextran sodium sulphate solution (DSS). The degree of severity of the illness was established on the basis of a disease activity index (DAI). This consisted of the daily documentation of body weight loss, stool consistency, rectal bleeding, food and water intake, and is defined in the publication “Bank, U., Heimburg, A., Helmuth, M., Stefin, S., Lendeckel, U., Reinhold, D., Faust, J., Fuchs, P., Sens, B., Neubert, K., Täger, M., Ansorge, S.; Triggering endogenous immunosuppressive mechanisms by combined targeting of dipeptidyl peptidase IV (DPIV/CD26) and aminopeptidase N (APN/CD13)—A novel approach for the treatment of inflammatory bowel disease; International Immunopharmacology 6: 1925-1934 (2006)”.

Regulatory T-cells from peripheral venous blood were recovered in parallel by means of density-gradient centrifugation and magnetic separation using CD25 Microbeads.

On day 3, either the aminopeptidase N inhibitor phebestin (0.5 mg/kgKG), untreated CD4⁺CD25⁺ Treg cells (1 million cells/animal) or activated CD4⁺CD25⁺ Treg cells (1 million cells/animal) were applied once intravenously.

The activation of the Treg cells occurred ex situ using the method according to the invention by incubating the Treg cells for 45 minutes in the presence of phebestin [500 μg/ml].

While the single application of untreated regulatory T-cells or phebestin intravenously had no effect on the activity of the disease (measured using the disease activity index), the single administration of the Treg cells activated by activation with alanyl-aminopeptidase inhibitor resulted in a significant reduction in the clinical symptoms of the illness up to 48 hours after the application (p<0.05).

1.-38. (canceled)

39. A method for activating regulatory T-cells (Treg cells) of the human or animal body, wherein the method comprises bringing the Treg cells in a suitable liquid medium into contact with at least one inhibitor selected from inhibitors of alanyl-aminopeptidase (aminopeptidase N; APN) and inhibitors of peptidases with the same substrate specificity to induce a suppressive effect of the Treg cells.

40. A method for the ex-vivo activation of regulatory T-cells (Treg cells) of the human or animal body, wherein the method comprises:
(a) recovering at least one body fluid comprising Treg cells from at least one body selected from human and animal bodies;
(b) isolating the Treg cells from the at least one body fluid and purifying the Treg cells;
(c) bringing the isolated and purified Treg cells in a suitable fluid medium into contact with at least one inhibitor selected from inhibitors of alanyl-aminopeptidase (aminopeptidase N; APN) and inhibitors of peptidases with the same substrate specificity for a period which is sufficient for activating the Treg cells; and
(d) returning the thus treated Treg cells in a suitable medium into at least one human or animal body.

41. The method of claim 39, wherein the at least one inhibitor comprises at least one compound selected from actinonin, leuhistin, phebestin, amastatin, bestatin, probestin, arphamenin A, arphamenin B, MR 387 A, MR 387 B, β-aminothiols, α-aminophosphinic acids and esters and salts thereof, α-aminophosphonates, α-aminoboric acids, α-aminoaldehydes, hydroxamates of α-amino acids, N-phenylphthalimides, N-phenylhomophthalimides, α-keto amides, thalidomide and derivatives thereof.

42. The method of claim 41, wherein the at least one inhibitor comprises at least one compound selected from α-keto amides, α-aminophosphinic acids, N-phenylhomophthalimides, α-aminophosphonates, and phebestin.

43. The method of claim 42, wherein the at least one inhibitor comprises at least one compound selected from 3-amino-2-oxo-4-phenylbutyric acid amides, D-Phe-y[PO(OH)—CH₂]-Phe-Phe, PAQ-22, RB3014, and phebestin.

44. The method of claim 42, wherein the at least one inhibitor comprises at least one compound selected from PAQ-22, RB3014, and phebestin.

45. The method of claim 42, wherein the at least one inhibitor comprises at least PAQ-22.

46. The method of claim 39, wherein the at least one inhibitor comprises at least one compound selected from dual inhibitors of alanyl-aminopeptidase and of peptidases with the same substrate specificity and from dipeptidylpeptidases (IV) and of peptidases with the same substrate specificity from the group of compounds of formulae (1) and (2)
A-B-D-B′-A′ (1) and
A-B-D-E (2),
wherein A and A′ are the same or different and represent 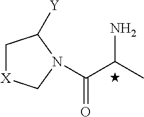
wherein X represents S, O, CH₂, CH₂CH₂, CH₂O or CH₂NH and Y represents H or CN and * represents a chiral carbon atom in the S- or L-configuration;
B and B′ are the same or different and represent an unsubstituted or substituted, unbranched or branched alkylene radical, cycloalkylene radical, aralkylene radical, heterocycloalkylene radical, heteroarylalkylene radical, aryl-amidoalkylene radical, heteroarylamidoalkylene radical, containing or not containing O, N or S, unsubstituted or mono- or polysubstituted arylene radical or heteroarylene radical with one or more five-, six- or seven-membered ring(s);
D represents —S—S— or —Se—Se—; and
E represents —CH₂—CH(NH₂)—R⁹ or —CH₂—*CH(NH₂)—R⁹, wherein R⁹ is an unsubstituted or substituted, unbranched or branched alkyl radical, cycloalkyl radical, aralkyl radical, heterocycloalkyl radical, heteroarylalkyl radical, arylamidoalkyl radical, heteroarylamidoalkyl radical, containing or not containing O, N or S, unsubstituted or mono- or polysubstituted aryl radical or heteroaryl radical with one or more five-, six- or seven-membered ring(s), and * represents a chiral carbon atom in the S- or L-configuration;
and acid addition salts thereof with organic and/or inorganic acids.

47. The method of claim 46, wherein the acid addition salts of the compounds of formulae (1) or (2) are selected from hydrochlorides, trifluoroacetates, tartrates, succinates, formiates, and citrates.

48. The method of claim 46, wherein the at least one inhibitor comprises at least one compound of formula (1a): 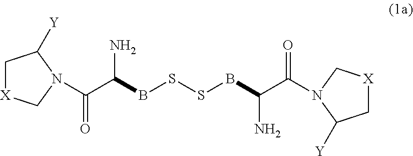
and/or an acid addition salt thereof.

49. The method of claim 48, wherein the compound of formula (la) comprises at least one compound wherein X, Y and B have the following meanings:

| | | | | Empirical |
|---|---|---|---|---|
| No. | B | X | Y | Formula |
| I | —CH₂— | —CH₂— | H | C₁₄H₂₆N₄O₂S₂ |
| II | —CH₂— | S | H | C₁₂H₂₂N₄O₂S₄ |
| III | —CH₂— | —CH₂— | CN | C₁₆H₂₄N₆O₂S₂ |
| IV | 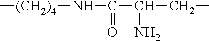 | S | H | C₂₄H₄₆N₈O₄S₄ |
| V | 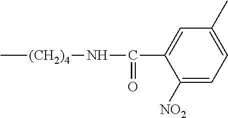 | S | H | C₃₂H₄₂N₈O₈S₄ |
| VI | 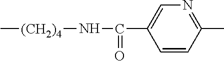 | S | H | C₃₀H₄₂N₈O₄S₄ |

and/or an acid addition salt thereof.

50. The method of claim 46, wherein the at least one inhibitor comprises at least one compound of formula (2a): 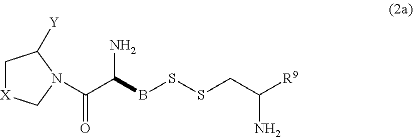
and/or an acid addition salt thereof.

51. The method of claim 50, wherein the compound of formula (2a) comprises at least one compound wherein X, Y, R⁹ and B have the following meanings:

| No. | B | R⁹ | X | Y | Empirical Formula |
|---|---|---|---|---|---|
| VII | —CH₂— | 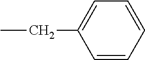 | S | H | C₁₅H₂₃N₃OS₃ |
| VIII | 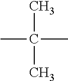 | 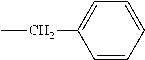 | S | H | C₁₇H₂₇N₃OS₃ |
| IX | 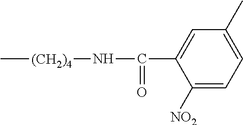 | 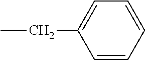 | S | H | C₂₅H₃₃N₅O₄S₃ |
| X | 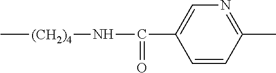 | 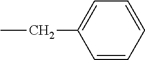 | S | H | C₂₄H₃₃N₅O₂S₃ |
| XI | 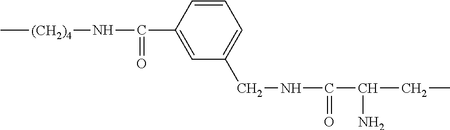 | 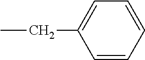 | S | H | C₂₉H₄₂N₆O₃S₃ |
| XII | 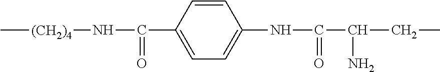 | 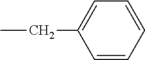 | S | H | C₂₆H₄₀N₆O₃S₃ |
| XIII | 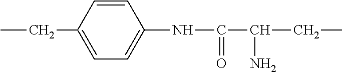 | 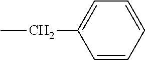 | S | H | C₂₄N₃₃N₅O₂S₃ |

and/or an acid addition salt thereof.

52. The method of claim 39, wherein the method comprises an additional use of at least one of a peptide fragment from a pathogenic autoantigen or a synthetic analogue and a specific antigenic component of a pathogenic microorganism.

53. The method of claim 52, wherein at least one of MBP (myelin basic protein), MOG (myelin oligodendrocyte glycoprotein), MAG (myelin associated glycoprotein) and PLP (proteolipid protein) is used as a peptide fragment from a pathogenic autoantigen and/or wherein at least one of a coat protein and a membrane glycolipid complex is used as a specific antigenic component of a pathogenic microorganism.

54. The method of claim 39, wherein the Treg cells are isolated from one or more of blood, fractions thereof, lymph, exudates and local compartments.

55. The method of claim 54, wherein the Treg cells are isolated from one or more of peripheral blood, pleura and peritoneum.

56. The method of claim 39, wherein the isolated regulatory T-cells are brought into contact with the at least one inhibitor in a liquid which comprises at least one liquid selected from physiologically acceptable solutions, cell culture media and nutrient media.

57. The method of claim 39, wherein the Treg cells are returned into at least one human or animal body by at least one of intravenous application, intra-arterial application, intracavitary application, intrathecal application and intradermal application.

58. The method of claim 39, wherein the Treg cells are incubated with the at least one inhibitor in at least one of a customary cell culture vessel, a culture dish, a culture plate, a cell culture reactor, a cell culture flask, a cell culture bag, a dual- or multi-chambered system suitable for cell cultures, and a hollow fiber reactor.

59. Activated Treg cells which are obtainable by the method of claim 39.

60. A method of preventing, alleviating or treating a condition, wherein the method comprises administering to a patient in need thereof the activated Treg cells of claim 59 in an amount sufficient to prevent, alleviate or treat the condition and wherein the condition comprises one or more of
an autoimmune disorder,
at least one of an allergy, bronchial asthma, and a chronic obstructive lung disease (COPD),
a disease of chronic-inflammatory genesis,
at least one of a neuronal disease and brain damage,
a skin disease,
a fibrose,
at least one of a tumor disease and a sepsis,
at least one of multiple sclerosis, Crohn's disease and ulcerative colitis,
an inflammatory disease,
bronchial asthma,
at least one of a skin and a mucous membrane disease,
an acute neuronal disease,
a chronic neuronal disease,
at least one of a prion-related disease condition and amyotrophic lateral sclerosis,
at least one of atherosclerosis, arterial inflammation, and stent restenosis,
at least one of a tumor, a metastase, and prostate cancer,
severe acute respiratory syndrome (SARS),
a sepsis or a sepsis-like condition,
type II diabetes.
